(19) United States
(12) Patent Application Publication
(10) Pub. No.: US 2024/0368586 A1
(43) Pub. Date: Nov. 7, 2024

(54) GUIDE RNA SEQUENCING CONFIRMATION

(71) Applicant: Synthego Corporation, Redwood City, CA (US)

(72) Inventors: Alex Campbell, Redwood City, CA (US); Sultana Jahan, Redwood City, CA (US); Laura Donohue, Redwood City, CA (US); Pedro Mendez, Redwood City, CA (US); Puzhou Wang, Redwood City, CA (US); Brendan Galvin, South San Francisco, CA (US)

(21) Appl. No.: 18/655,927

(22) Filed: May 6, 2024

Related U.S. Application Data
(60) Provisional application No. 63/500,510, filed on May 5, 2023.

(51) Int. Cl. C12N 15/10; C12Q 1/6806; C12Q 1/686; C12Q 1/6874

(52) U.S. Cl. CPC C12N 15/1096; C12Q 1/6806; C12Q 1/686; C12Q 1/6874

(57) ABSTRACT

Provided herein are methods for assessing a preparation of guide RNA molecules for quality, including for purity and identity.

### CROSS-REFERENCE TO RELATED APPLICATIONS

This application claims the benefit of priority under 35 U.S.C. § 119(e) of U.S. Provisional Application No. 63/500,510, filed May 5, 2023, the contents of which are incorporated herein by reference in its entirety.

### SEQUENCE LISTING

The contents of the electronic sequence listing (File Name: 17477400176_SL_ST26.xml; Size: 48,457 bytes; Date of Creation: Jun. 13, 2024) submitted herewith is herein incorporated by reference in its entirety.

### BACKGROUND

Clustered regularly interspaced short palindromic repeats (CRISPR)/CRISPR-associated (Cas) (“CRISPR/Cas”) systems can be used as a gene editing tool in a plethora of different organisms to generate breaks at a target site and subsequently introduce mutations at the locus. Two main components can be needed for the gene editing process: an endonuclease like Cas enzyme and a short RNA molecule to recognize a specific DNA target sequence. Instead of engineering a nuclease enzyme for every DNA target, the CRISPR/Cas system can rely on customized short RNA molecules to recruit the Cas enzyme to a new DNA target site. Such short RNA molecules are generally referred to as guide RNAs.

The CRISPR/Cas system can be used in prokaryotic and eukaryotic systems for genome editing and transcriptional regulation. There is significant therapeutic potential for CRISPR/Cas systems. In some cases, however, the CRISPR/Cas system can yield unwanted off-target genome editing and varied editing efficiency across different gene targets. Accordingly, regulatory authorities require heightened quality and safety criteria for potential CRISPR/Cas therapeutics, including the purity of the guide RNAs.

Currently, regulatory agencies require the assessment of the purity of guide RNA lots using electrospray ionization (ESI) and high-performance liquid chromatography (HPLC). However, those methods have two major limitations: (i) the sensitivity in detecting impurities is relatively low (1-2%), and (ii) those methods can't resolve the sequence composition of single-guide RNA molecules. More importantly, the draft regulations provided in 2020 by the World Health Organization with the procedures required to evaluate the quality, safety, and efficacy of mRNA vaccines indicated a preference under subheading 6.4.4.1 for selecting a method to address identity and purity in determining the sequence of the entire mRNA.

Some methods are being used to sequence single-guide RNA molecules for quality control. For example, Editas at TIDES on May 10, 2018, described a next-generation sequencing (NGS) method for guide RNA quality control, however that method is prone to mis-priming and generation of artifacts that interfere with reliable outcomes.

There is a need in the art for methods of sequencing guide RNAs that overcome the limitations of current methods.

### SUMMARY

Disclosed herein are methods for determining the sequence of an RNA molecule, for example to confirm the RNA molecule's sequence relative to a reference sequence, including the use of a modified template switching oligonucleotide (TSO) as described herein.

In one aspect, methods are disclosed herein for determining a sequence of a guide RNA to confirm the sequence relative to a reference guide RNA sequence. In certain aspects, the methods include: providing a guide RNA from a sample; preparing a first and second strand mix including: (i) the guide RNA, (ii) DNA primer that hybridizes to the 3′ end of the guide RNA, (iii) dNTPs, and (iv) a template switching oligonucleotide (TSO) including a modification at the 3′ terminal end; incubating the first strand mix and the second strand mix under conditions to allow synthesis of cDNA. In certain aspects, the methods also include purifying the cDNA, amplifying the cDNA, purifying the amplified cDNA; and sequencing the amplified cDNA.

The disclosure also provides methods for assessing the identity of a guide RNA in a sample, including: providing a guide RNA from the sample; preparing an RNA ligation mix including: the guide RNA, 3′ an adapter molecule (i.e., adapter oligonucleotide) containing a universal priming sequence, and RNA ligase; preparing a first and second strand mix including: the ligated guide RNA, DNA primer that hybridizes to the 3′ end of the adapter molecule or the guide RNA, dNTPs, and a template switching oligonucleotide (TSO) including a modification at the 3′ end; incubating the first strand mix and the second strand mix under conditions to allow synthesis of cDNA; sequencing the amplified cDNA; comparing the sequence of the amplified cDNA to a reference guide RNA sequence.

Also disclosed herein are methods for preparing a cDNA copy of a guide RNA, including: providing a guide RNA from a sample; preparing a first and second strand mix including: the guide RNA; DNA primer that hybridizes to the 3′ end of the guide RNA; dNTPs; a template switching oligonucleotide (TSO) including modification at the 3′ end; a reverse transcriptase; and incubating the first strand mix and the second strand mix under conditions to allow synthesis of cDNA.

The disclosure provides a cDNA library preparation mixture, including a guide RNA, a DNA primer that hybridizes to the 3′ end of the guide RNA, and a template switching oligonucleotide (TSO) including a modification at the 3′ end that prevents DNA polymerase activity.

In certain aspects, the modification is a 3-dideoxycytosine (3ddC) added to the 3′ end of the TSO.

In certain aspects, the cDNA library preparation further includes dNTPs and a reverse transcriptase.

In particular aspects, the cDNA library preparation is subjected to conditions suitable for polymerase chain reaction to occur to generate a first and second strand of cDNA from the guide RNA template.

Also provided herein are template switching oligonucleotides including a 3′ end modification. In certain aspects, the TSO modification at the 3′ end inhibits DNA polymerase activity (i.e., prevents 3′ elongation (extension) of DNA by a DNA polymerase). In one aspect, the TSO modification is a 3-Dideoxycytosine (3ddC) or other dideoxynucleotide added to the 3′ end of the TSO.

In other aspects, the 3′ end of the TSO includes the sequence -GGG-3ddC, -rGrGrG-3ddC, -GGGG-3ddC or -GrGrGrG-3ddC, wherein the 3ddC is at the 3′ terminal end.

In some aspects, a universal adapter oligonucleotide is ligated to the guide RNA providing a 3 end priming site for PCR.

### BRIEF DESCRIPTION OF THE DRAWINGS

FIG. 1 illustrates a method for generating cDNA from a single-guide RNA (sgRNA) using a template-switching oligonucleotide (TSO) that hybridizes to a tri-cytosine sequence (i.e., 3′-CCC-) added by a reverse transcriptase during first strand synthesis. As shown on the right side of the figure, when an internal tri-cytidine sequence is present within the sgRNA, mis-priming can occur as the TSO hybridizes with the internal -CCC- sequence instead of the 3′-CCC-. Thus, artifacts are produced among the full length cDNAs.

FIG. 2 illustrates a solution to the mis-priming problem observed in FIG. 1. TSOs including a modification at the 3′ end are used that block DNA polymerase extension. Thus, only full length cDNA molecules are generated.

FIG. 3 illustrates the universal adapter approach with an adapter oligonucleotide containing a cDNA priming sequence ligated to the 3′ end of the guide molecule.

FIG. 4 shows a scatterplot of recovered percentages of SNV and RelA species based on UMI correction against their expected percentages.

FIG. 5 shows the distribution of unique read/UMI combination percentages in all samples with 30% molar percentage.

FIG. 6A shows the recovered percentages based on UMI correction in simulated samples with expected percentages of 30%.

FIG. 6B shows the recovered percentages based on UMI correction in simulated samples with expected percentages of 80%.

FIG. 7 shows BioAnalyzer electropherograms of a cDNA library with TSO phosphorothioate (TSO-PTA), TSO with three carbon spacers (TSO-3ddC) and TSO with didieoxyC (TSO-ddC).

FIG. 8 shows the dominant species and their full-length sequences aligned to the RelA reference sequence. Only sequences that included greater than 1% of total sequencing read lengths are shown. Species are ordered by abundance of sequencing reads. Percent of total reads is listed in the column on the right. Base pair deletions and truncations are shown as dashes aligned to the reference. Sequences shown, from top to bottom, are: SEQ ID NO: 21, SEQ ID NO: 22, SEQ ID NO: 21, SEQ ID NO: 23, SEQ ID NO: 24, SEQ ID NO: 21, SEQ ID NO: 21, SEQ ID NO: 25, SEQ ID NO: 26, SEQ ID NO: 27.

FIG. 9A shows bioanalyzer traces from TSOv1 (top) and TSOv2 (bottom). TSO v1 had 89% full length product in the larger peak with 11% small, truncated peaks. TSOv2 had 100% full length product shown as a single peak.

FIG. 9B shows the dominant species and their full length sequences aligned to the DGK guide for TSOv1 (top) and TSOv2 (bottom). Only sequences that included greater than 1% of total sequencing read lengths are shown. Species are ordered by abundance of sequencing reads. Percent of total reads is listed in the column on the right. Base pair deletions and truncations are shown as dashes aligned to the reference. Mismatching bases are shown in different color aligned to the reference. Sequences shown, from top to bottom, are: SEQ ID NO: 28, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 28, SEQ ID NO: 28, SEQ ID NO: 35, SEQ ID NO: 34, SEQ ID NO: 36, SEQ ID NO: 33.

FIG. 10A shows the dominant species and their full-length sequences aligned to the RelA reference sequence using the universal approach. Only sequences that included greater than 1% of total sequencing read lengths are shown. Species are ordered by abundance of sequencing reads. Percent of total reads is listed in the column on the right. Base pair deletions and truncations are shown as dashes aligned to the reference. Mismatching bases are shown in different color aligned to the reference. Sequences shown, from top to bottom, are: SEQ ID NO: 21, SEQ ID NO: 21, SEQ ID NO: 37, SEQ ID NO: 27, SEQ ID NO: 38.

FIG. 10B shows the dominant species and their full-length sequences aligned to the ACM360 reference sequence using the universal approach. Only sequences that included greater than 1% of total sequencing read lengths are shown. Species are ordered by abundance of sequencing reads. Percent of total reads is listed in the column on the right. Base pair deletions and truncations are shown as dashes aligned to the reference. Mismatching bases are shown in different color aligned to the reference. Sequences shown, from top to bottom, are: SEQ ID NO: 39, SEQ ID NO: 39, SEQ ID NO: 40, SEQ ID NO: 41, SEQ ID NO: 42, SEQ ID NO: 43, SEQ ID NO: 44.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

The present invention is described herein using several definitions, as set forth below and throughout the application.

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Finally, as used herein any reference to “an embodiment”, “one embodiment” or “some embodiments” means that a particular element, feature, structure, or characteristic described in connection with the embodiment is included in at least one embodiment disclosed herein. The appearances of the phrase “in some embodiments” in various places in the specification are not necessarily all referring to the same embodiment, and embodiments may include one or more of the features expressly described or inherently present herein, or any combination of sub-combination of two or more such features, along with any other features which may not necessarily be expressly described or inherently present in the instant disclosure.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition.

As used herein, all percentages are percentages by weight, unless stated otherwise.

As used herein and unless otherwise indicated, the terms “a” and “an” are taken to mean “one”, “at least one” or “one or more”.

The term “polynucleotide” or “nucleic acid,” as used interchangeably herein, can generally refer to a polymeric form of nucleotides of any length, either ribonucleotides and/or deoxyribonucleotides. Thus, these terms include, but are not limited to, single-, double-, or multi-stranded DNA or RNA, genomic DNA, complementary DNA (cDNA), guide RNA (gRNA), messenger RNA (mRNA), DNA-RNA hybrids, or a polymer including purine and pyrimidine bases or other natural, chemically or biochemically modified, non-natural, or derivatized nucleotide bases.

The term “oligonucleotide,” as used herein, can generally refer to a polynucleotide of between about 5 and about 100 nucleotides of single- or double-stranded DNA or RNA. However, for the purposes of this disclosure, there may be no upper limit to the length of an oligonucleotide. In some cases, oligonucleotides can be known as “oligomers” or “oligos” and can be isolated from genes, or chemically synthesized by methods known in the art. The terms “polynucleotide” and “nucleic acid” should be understood to include single-stranded (such as sense or antisense) and double-stranded polynucleotides.

Sequences of polynucleotides and oligonucleotides presented herein are presented left to right in the 5′ to 3′ order unless otherwise indicated.

The term “modified nucleotide,” as used herein, can generally refer to a nucleotide having a modification to the chemical structure of one or more of the base, the sugar, and/or the phosphodiester linkage or backbone portions, including the nucleotide phosphates, relative to a naturally occurring base, sugar, and/or phosphodiester linkage or backbone portions.

The term “hybridization” or “hybridizing,” as used herein, can generally refer to a process where completely or partially complementary polynucleotide strands come together under suitable hybridization conditions to form a double-stranded structure or region in which the two constituent strands are joined by hydrogen bonds. In some cases, modified nucleotides can form hydrogen bonds that allow or promote hybridization. In some cases, a guanine (G) of a protein-binding segment of a subject DNA-targeting RNA molecule can be considered complementary to a uracil (U), and vice versa.

### Assessment Methods

Disclosed herein are methods for sequencing full-length guide RNAs and RNA molecules of various lengths. In some embodiments, the methods are useful for sequencing multiple types of guide RNAs (e.g., 100-mer, 119-mer) with error correction to less than 1%. In certain embodiments, the methods include generating cDNA copies of particular guide RNAs in a composition and sequencing the cDNA to determine the quality of the guide RNA composition in terms of its purity, for example to determine the percent of guide RNAs in the composition that have a particular sequence relative to a reference guide RNA sequence. Sequencing is accomplished, for example, 10 by next-generation sequencing (NGS) or Sanger sequencing. NGS is well known to those of skill in the art and is described, for example, in Shendure et al., 2008, Nat Biotechnol., 26(10):1135-1145 and Schuster, 2008, Nat Methods, 5(1):16-18. Sanger and other common sequencing techniques are described, for example, in Heather and Chain, 2016, Genomics, 107(1): 1-8.

In certain embodiments, a “UMI error correction” can be used to remove PCR and 15 sequencing bias. UMIs can be added, for example, during the reverse transcription step of a method disclosed herein. Alternatively, UMIs can be added during the ligation step of a method disclosed herein. In some embodiments, the UMIs can be added to the 3′-end of RNA sequences. In some embodiments, the UMI sequences are at least 10 nucleotides in length. In some embodiments, the UMI sequences are at least 21 nucleotides in length. In some embodiments, the length of UMI sequences is determined based on the concentration of RNA in a sample. In some embodiments, the method can include determining the percentage of recovered RNA species based on the plurality of RNA species based on the number of UMI sequences. In some embodiments, the method can include determining the percentage of each UMI sequence with unique RNA sequence reads. In some embodiments, the number of UMI sequences is about the number of RNA molecules in the sample. Those of skill in the art regularly use UMIs to analyze sequencing errors. Such procedures are described in the art as described, for example, in Smith et al., 2017, Genome research, 27.3:491-499.

Provided herein are methods for identifying impurities in a guide RNA composition or preparation.

As used herein, “impurities” include any sequence variation of a polynucleotide sequence relative to a predetermined sequence. In certain embodiments, the predetermined sequence is the sequence of a reference polynucleotide sequence, e.g., a reference guide RNA sequence. The sequence variation can be, for example, a truncation, nucleotide insertion, nucleotide deletion, and/or nucleotide substitution relative to the predetermined sequence (e.g., reference guide RNA sequence).

Provided herein are methods for determining a sequence of a guide RNA, including: providing a guide RNA from a sample, preparing a first and second strand mix, incubating the first and second strand mix under conditions to allow synthesis of cDNA, wherein the mixture of first 10 and second strand mixes include the guide RNA, dNTPs, a reverse transcriptase, and a template switching oligonucleotide (TSO) including a 3′ end modification.

Methods disclosed herein can be used to generate cDNA using single guide RNA (sgRNA) as a template. In some embodiments, the first strand can be synthesized using a tailed primer that hybridizes with nucleotides of the 3′ end of the sgRNA. For the second strand synthesis, a template-switching oligonucleotide (TSO) with a 3′ end modification (e.g., a 3-Dideoxy Cytosine (3ddC) modification) can be used as described herein.

As the priming site for cDNA synthesis typically includes the 3′ end of the sgRNA scaffold, sgRNA molecules with a potential truncation in those last several (e.g., 17) nucleotides at the 3′ end might not be transformed into cDNA and will not be analyzed. In some instances, a sgRNA molecule is hydrolyzed from the 3′ end and will be missing a portion of the reverse transcriptase priming site, thus will not be sequenced. To address these situations, methods disclosed herein can include ligation of a linker oligonucleotide to the 3′ end of the guide RNA using RNA ligase. The ligation-based method overcomes any bias against 3′ hydrolysis or truncated molecules.

As some guides for nucleases, such as but not limited to Cas12a, contain a targeting region at the 3′ end of the guide, designing a cDNA priming site for each variable region of a synthesized guide is not feasible. To address this situation, methods are disclosed herein that include ligation of an adapter oligonucleotide to the 3′ end of the guide using RNA ligase. This ligation-based method bypasses the need for designing multiple 3′ cDNA primers based on the sequence of the RNA molecule to be sequenced.

In one embodiment, the adapter oligonucleotide ligated to the 3′ end of an RNA molecule, e.g., a guide RNA, includes a priming site for a primer to generate a cDNA library. The adapter oligonucleotide can also include a sequence or sequences useful for NGS sequencing process as understood by those of skill in the art. An adapter oligonucleotide for ligating to an RNA molecule, in particular a guide RNA, is also referred to herein as a universal adapter oligonucleotide.

The universal adapter oligonucleotide can be, for example, DNA or a combination of DNA and RNA and can include a modified base at the discretion of those carrying out a method of the disclosure. The adapter oligonucleotide can be any length depending on the RNA molecule being sequenced. Certain non-limiting examples of adapter oligonucleotides are described herein and are available from various commercial vendors as known by those of skill in the art. Adapter oligonucleotides can be designed by those of skill in the art for cDNA library preparation and NGS sequencing.

In certain embodiments, an adapter oligonucleotide has a 3′ end modification that prevents extension activity of a DNA polymerase, including but not limited to 3ddC and other dideoxynucleotides.

As guides for some nucleases in such families as Cas12 have a shorter length than others, traditional cDNA library preparation methods may not be feasible as these shorter guide RNAs will be removed during purification steps. To address this situation, methods are disclosed herein that include ligation of an adapter oligonucleotide of a length of about 30 nucleotides or more to 20 the guide RNA molecule. The ligation of a long linker molecule can bypass the bias of next generation sequencing library preparation against shorter library molecules.

Non-limiting examples of suitable adapter oligonucleotide sequences for ligating to the 3′ end of the guide RNA include:

TABLE 1
examples of suitable adapter oligonucleotide sequences for
ligating to the 3′ end of the guide RNA.

| | SEQ ID |
|---|---|
| Oligonucleotide | NO: |
| 5′-NNCTGTCTCTTATACACATCTCCGAGCCCACGAGAC | 1 |
| 5′-NNCTGTCTCTTATACACATCTCCGAGCCCACGAGAC3ddC | 2 |
| 5′-CTGTCTCTTATACACATCTCCGAGCCCACGAGAC | 3 |
| 5′-CTGTCTCTTATACACATCTCCGAGCCCACGAGAC3ddC | 4 |
| 5′- | 5 |
| NNATCACATGTCAGTCATCTACGATCGATCATCTGTCTCTTATACACATCTCCGAGC | |
| CCACGAGAC | |

PCR can be used to create a library of cDNAs for determining the sequence of guide RNA as described herein. PCR (polymerase chain reaction) is a well-known technique for amplifying DNA molecules and making many copies of a specific region of a DNA. PCR uses DNA polymerase and small fragments of DNA called “primers” designed to hybridize to specific portions of a target DNA sequence. In the presence of dNTPs (DNA nucleotide bases) the polymerase adds the DNA bases to the primers thereby extending a new strand of DNA complementary to the DNA molecule or DNA region of interest. The process can be repeated as 10 desired, each time doubling the number of DNA copies being generated. PCR reagents and kits are widely available commercially and used in a variety of laboratory settings.

In some embodiments, a method of the disclosure includes: a) providing a guide RNA from a sample; b) preparing a first and second strand mix including: the guide RNA, DNA primer that hybridizes to a particular sequence of the 3′ end of the guide RNA, dNTPs, a template switching oligonucleotide (TSO) including a 3′ end modification, and a reverse transcriptase; c) incubating the first strand mix and the second strand mix under conditions to allow synthesis of cDNA; d) purifying the cDNA; e) amplifying the cDNA; f) purifying the amplified cDNA; and g) sequencing the amplified cDNA.

In one embodiment, the 3′end modification on the TSO prevents second strand extension 20 during PCR amplification. In a particular embodiment, the 3′ end modification is a 3-Dideoxycytosine (3ddC) located at the 3′ terminus of the TSO as described herein. 3ddC is also known as 3′-Dideoxy-C(3′ddC) and is known to prevent 3′ extension by DNA polymerases. Besides 3′ddC, any other end modification that can prevent DNA polymerase extension can be used in methods disclosed herein, including for example 3′ inverted dT, 3′ C3 spacer, 3′ amino, and 3′ phosphorylation. In certain embodiments, the modification is a dideoxynucleotide (e.g., ddC, ddG, ddT, or ddA).

In some embodiments, a method includes the step of ligating an adapter molecule to the guide RNA that includes a particular sequence for sequencing purposes as understood by those of skill in the art and described, for example, herein. Such adapters are oligonucleotides that can be attached to sequences during sequencing library preparation that enable the cDNA products to be 10 immobilized, for example, on beads or a flow-cell, for sequencing of the cDNA. Various adapters can be used and a number of them are available commercially. For example, the p5 and p7 adapters from Illumina are commonly used for next-generation sequencing (NGS) systems such as those available from Illumina, Inc. (San Diego, CA). Such adapters can be included in or as a universal adapter oligonucleotide in a method disclosed herein.

Ligating an adapter can be accomplished, for example, by any method known to those of skill in the art using ligases, such as covalent attachment.

In some embodiments, the sequence of amplified cDNA prepared in a method of the disclosure is compared with a reference guide RNA sequence to check the quality of guide RNA synthesis.

### CRISPR

The term “CRISPR/Cas,” as used herein, refers to a ribonucleoprotein (“RNP”) complex including a guide RNA (gRNA) and a CRISPR-associated (Cas) endonuclease. The term “CRISPR” refers to the Clustered Regularly Interspaced Short Palindromic Repeats and the related 25 system thereof. While CRISPR was discovered as an adaptive defense system that enables bacteria and archaea to detect and silence foreign nucleic acids (e.g., from viruses or plasmids), it can be adapted for use in a variety of cell types to allow for polynucleotide editing in a sequence-specific manner. In some cases, one or more elements of a CRISPR system can be derived from a type I, type II, or type III CRISPR system. In the CRISPR type II system, the guide RNA can interact with Cas and direct the nuclease activity of the Cas enzyme to a target region. The target region can include a “protospacer” and a “protospacer adjacent motif” (PAM), and both domains can be needed for a Cas enzyme mediated activity (e.g., cleavage). The protospacer can be referred to as a target site (or a genomic target site). The gRNA can pair with (or hybridize) the opposite strand of the protospacer (binding site) to direct the Cas enzyme to the target region. The PAM site generally refers to a short sequence recognized by the Cas enzyme and, in some cases, required for the Cas enzyme activity. The sequence and number of nucleotides for the PAM site can differ depending on the type of the Cas enzyme.

As used herein, the term “target site” also refers generally to a nucleic acid sequence to which a binding molecule will bind under appropriate conditions, for example in a gene of interest. In some embodiments, a target site is the nucleic acid sequence to which a guide RNA as provided herein will bind.

The term “Cas,” as used herein, can generally refer to a wild type Cas protein, a fragment thereof, or a mutant or variant thereof. A Cas protein can include a protein of or derived from a CRISPR/Cas type I, type II, or type III system, which can be an RNA-guided polynucleotide-binding or nuclease activity. Examples of suitable Cas proteins include CasX, Cas3, Cas4, Cas5, Cas5e (or CasD), Cas6, Cas6e, Cas6f, Cas7, Cas8a1, Cas8a2, Cas8b, Cas8c, Cas9 (also known as Csn1 and Csx12), Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, homologues thereof, and modified versions thereof. In some cases, a Cas protein can include a protein of or derived from a CRISPR/Cas type V or type VI system, such as Cpf1 (Cas12a), C2c1 (Cas12b), C2c2, homologues thereof, and modified versions thereof. In some cases, a Cas protein can be a catalytically dead or inactive Cas (dCas).

### Guide RNAs

The term “guide RNA” or “gRNA,” as used herein, can generally refer to an RNA molecule (or a group of RNA molecules collectively) that can bind to a Cas protein and aid in targeting the Cas protein to a specific location within a target polynucleotide (e.g., a DNA). A guide RNA can include a CRISPR RNA (crRNA) segment and a trans-activating crRNA (tracrRNA) segment. The term “crRNA” or “crRNA segment,” as used herein, can refer to an RNA molecule or portion thereof that includes a polynucleotide-targeting guide sequence, a stem sequence, and, optionally, a 5′-overhang sequence. crRNA is described, for example, by Jiang et al. (Nat Biotechnol. 2013 March; 31(3): 233-239) and Jinek et al. (2012, Science, 337:816-821). The term “tracrRNA” or “tracrRNA segment,” can refer to an RNA molecule or portion thereof that includes a protein-binding segment (e.g., the protein-binding segment is capable of interacting with a CRISPR-associated protein, such as a Cas9). The term “guide RNA” as used herein encompasses a single guide RNA (sgRNA), where the crRNA segment and the tracrRNA segment are located in the same RNA molecule as described, for example, by Jinek et al. (2012, Science, 337:816-821). The terms sgRNA and single polynucleotide sgRNA are used interchangeably herein.

In some embodiments, a guide RNA provided herein includes a scaffold domain and a targeting domain. The “scaffold” domain is the portion of the guide RNA that contains a nucleic acid sequence that interacts with a Cas enzyme (e.g., tracrRNA) while the “targeting domain” contains a nucleic acid sequence specific to the target site of the target polynucleotide (e.g., crRNA). The targeting domain will vary from guide to guide depending on the specific sequence to be targeted. Thus, the targeting domain is variable. For example, in a 100 nucleotide sgRNA, the scaffold is 80 nucleotides, and the targeting domain is 20 nucleotides. The scaffold is located at the 3′ end of the sgRNA in this example. One of skill in the art will recognize the scaffold and targeting domains can have varying lengths depending on the nature of the target sequence and the particular CRISPR Cas nuclease to be used in the selected CRISPR/Cas system.

In some embodiments, a sgRNA sequenced in a method disclosed herein includes a crRNA as the targeting domain and a tracrRNA as the scaffold domain.

In certain embodiments, methods disclosed herein can be used to sequence both the scaffold and the targeting domains of a guide RNA in one continuous sequencing method.

Designing gRNA Oligonucleotides

In one embodiment, the present disclosure discloses methods for sequencing guide RNAs intended for hybridizing to a genomic region of interest in a cell. The genomic region of interest can be a gene of a genome of a species. The gRNA oligonucleotides can be generated by selecting a transcript from a plurality of transcripts of the gene. The method can include identifying an initial gRNA or set of gRNAs that hybridize different target sites in the gene of the selected transcript. The gene can be a gene of interest. The genomic region of interest can be a non-coding region of the genome. The non-coding region can be a regulatory element. The regulatory element can be a cis-regulatory element or a trans-regulatory element. The cis-regulatory element can be a promoter, an enhancer, or a silencer.

Information including the genome of the species and/or a reference genome of the species can be obtained from a plurality of databases. In some cases, the plurality of databases can include gene and/or genome databases including sequencing data from DNA (DNA-seq) and/or RNA (RNA-seq). Examples of such genome databases include GENCODE, NCBI, Ensembl, {APPRIS}, and NIH Human Microbiome Project. Alternatively or in addition to, genomic information of an individual can be retrieved from personalized genome databases, including, but are not limited to, 23andMe, deCODE Genetics, Gene by Gene, Gene Planet, DNA Ancestry, uBiome, and healthcare providers. In some cases, necessary information including at least a portion of the genome of the species of interest can be provided by a user (e.g., via a user interface on a user device such as a personal computer).

The genome of the species can include some or a complete set of genetic material present in the species (e.g., cell or organism). Examples of the species include, but are not limited to, mammals (e.g., Homo sapiens, Mus musculus, Cricetulus griseus, Rattus norvegecus, Pan paniscus), fish (e.g., Danio rerio, Amphiprion frenatus), insects (e.g., Drosophila melanogaster), plants (e.g., Arabidopsis thaliana), roundworms (e.g., Caenorhabditis elegans), and microorganisms including bacteria (e.g., Escherichia coli, Lactobacillus bulgaricus). In some cases, the bacteria can include strains that are to be consumed by an individual as a supplement (e.g., in yogurt as a medium) and/or as a treatment (e.g., to suppress or ameliorate a condition). In some cases, the bacteria can include strains that are present in the body of an individual (e.g., human microbiome).

The genetic material of the genome can be DNA and/or RNA. The genetic material can include nucleic acid sequences in genes and intergenic regions. In some cases, the genetic material can be represented as a unit of a chromosome. In some cases, the genetic material can be represented as one or more transcripts that have been transcribed from a gene. The gene and its respective one or more transcripts can include one or more coding regions (i.e., exons). In some cases, the gene and its respective one or more transcripts can include one or more intragenic non-coding regions (i.e., introns). The one or more intragenic non-coding regions can be located between the coding regions. In some cases, a gene can encode one transcript. In some cases, a gene encodes a plurality of transcripts, each transcript having different variations of exons and introns from the gene. In an example, the RelA gene encodes for transcription factor p65, and the RELA gene of Homo sapiens encodes at least 18 known transcripts of varied length: RELA-202, RELA-207, RELA-226, RELA-205, RELA-201, RELA-208, RELA-220, RELA-207, RELA-215, RELA-204, RELA-222, RELA-213, RELA-225, RELA-211, RELA-219, RELA-221, and RELA-212. Thus, the plurality of transcripts can have different numbers of nucleotide bases (polynucleotide lengths). Alternatively or in addition to, the plurality of transcripts can be translated into polypeptides (e.g., proteins) having different numbers of amino acids (polypeptide lengths). In some cases, each of the plurality of transcripts can have different expression levels (abundance) reported relative to one or more other transcripts.

The gRNA or set of gRNAs can be designed to hybridize to a target region, also referred to herein as a binding site or target site. The target region can be in a gene or a portion of the gene in the genome of the species. In some cases, the portion of the gene can be an exon of the gene. The exon can be an exon found in each transcript of the gene. The exon can be the selected exon of a selected transcript of the plurality of transcripts of the gene from the aforementioned criteria. In some cases, one or more gRNAs in the initial set of gRNAs can be a single guide RNA (sgRNA). In some cases, the sgRNA can be a single polynucleotide chain. The sgRNA can include a hybridizing polynucleotide sequence (e.g., crRNA sequence) and a second polynucleotide sequence (e.g., tracrRNA sequence).

The hybridizing polynucleotide sequence can hybridize to the portion of the gene (e.g., the selected exon of the selected transcript of the plurality of transcripts of the gene). The hybridizing polynucleotide sequence of the sgRNA can range between 17 to 23 nucleotides. The hybridizing polynucleotide sequence of the sgRNA can be at least 17, 18, 19, 20, 21, 22, 23, or more nucleotides. The hybridizing polynucleotide sequence of the sgRNA can be at most 23, 22, 21, 20, 19, 18, 17, or less nucleotides. In an example, the hybridizing polynucleotide sequence of the gRNA is 20 nucleotides. The hybridizing polynucleotide sequence can be complementary or partially complementary to the target region. A hybridizing polynucleotide sequence complementary to the target region can include a sequence with 100% complementarity to a sequence of the target region. A gRNA partially complementary to the target region can include a sequence with at least 1, at least 2, at least 3, at least 4, or at least 5 mismatches relative to a sequence including 100% complementary to the target region. The hybridizing sequence is also referred to herein as a targeting sequence or targeting domain and is referred to by various names in the literature, including “crRNA” as described by Jiang et al. (Nat Biotechnol. 2013 March; 31(3): 233-239), for example.

A second polynucleotide sequence of the single polynucleotide chain sgRNA can interact (bind) with the Cas enzyme. The second polynucleotide sequence can be about 80 nucleotides. The second polynucleotide sequence can be 80 nucleotides. The second polynucleotide sequence can be at least 80, or more nucleotides. In some embodiments, the second polynucleotide sequence can be at most 80 or less nucleotides. The second polynucleotide sequence that interacts with the Cas enzyme is referred to by various names in the literature, including tracrRNA as described herein.

Overall, the single polynucleotide sgRNA can range between about 40 to about 120 nucleotides. The single polynucleotide chain sgRNA can be at least 40, 50, 60, 70, 80, 90, 97, 98, 99, 100, 101, 102, 103, 105, 110, 115, 116, 117, 118, 119, 120 or more nucleotides. The single polynucleotide chain sgRNA can be at most 103, 102, 101, 100, 99, 98, 97, or less nucleotides. In an example for Cas9 nuclease directed gene editing, the single polynucleotide chain sgRNA can be 100 nucleotides, wherein the hybridizing crRNA portion is 20 nucleotides and the Cas polypeptide interacting portion is 80 nucleotides.

### Quality Control Assessments

Disclosed herein are methods and systems for assessing a composition including guide RNA molecules for the presence of guide RNA impurities.

A “guide RNA impurity” can be, for example, a guide RNA variant having a sequence that differs from a reference guide RNA sequence, including truncations, substitutions, insertions, and/or deletions relative to a reference sequence.

In certain embodiments, criteria are provided for making a selection or to assess the quality of a guide RNA composition or a sample that includes guide RNAs based on the amount of guide RNA impurities present in the composition or sample.

The criteria and methods disclosed herein can be used, for example, to qualify a guide RNA composition for clinical and/or therapeutic development purposes (e.g., for demonstrating identity and purity in submissions to health authorities and/or to select guide RNA compositions for potential therapeutic development or further therapeutic development). The criteria and methods disclosed herein can also be used, for example, for selection of guide RNA compositions or samples for additional development purposes (e.g., for screening purposes to determine if a composition or sample is suitable for an intended purpose, such as for batch release or batch qualification in the manufacture of a drug substance or drug product including a guide RNA).

In some embodiments, a guide RNA composition is considered to have high purity (e.g., suitable for therapeutic development, release into commerce, and the like) if the composition includes at least about 50%, 60%, 70%, 80%, 85%, 90%, 95%, 96%, 97%, 98%, 99% or higher guide RNA molecules having the same sequence as a reference guide RNA sequence. In certain embodiments, a high purity composition of guide RNA has at least about 98%, 99%, 99.1%, 99.2%, 99.3%, 99.4%, 99.5%, 99.6%, 99.7%, 99.8%, or 99.9% guide RNAs with sequences that are identical to the reference guide RNA. A guide RNA is identical to the reference guide RNA where they have 100% identical nucleotide sequence alignment (i.e., there are no substitutions, 20 deletions, insertions, and/or truncations compared with the reference guide RNA sequence).

In certain embodiments, a composition is considered to have high purity if the composition includes:
(a) less than about 50%, 40%, 30%, 20%, 10%, 5%, 4%, 3%, 2%, 1%, 0.5%, or 0.1% of truncation variants including a truncation at a 5′ end, relative to a reference guide RNA sequence, (e.g., where there are 50% variants in a composition there are 50% guide RNAs identical to the references guide RNA sequence, where there are less than 40% variants in a composition there are more than 60% guide RNAs identical to the reference guide RNA sequence, where there are less than 5% variants in a composition there are more than 95% guide RNAs identical to the reference guide RNA sequence, and so forth); and/or
(b) less than about 50%, 40%, 30%, 20%, 10%, 5%, 1%, 0.5%, 0.3%, or 0.1% of sequence variants including a nucleotide substitution, insertion and/or deletion, relative to a reference guide RNA sequence (e.g., where there are 50% variants in a composition there are 50% guide RNAs identical to the references guide RNA sequence, where there are less than 40% variants in a composition there are more than 60% guide RNAs identical to the reference guide RNA sequence, where there are less than 5% variants in a composition there are more than 95% guide RNAs identical to the reference guide RNA sequence, and so forth); and/or
(c) a sequence variant present at a level greater than or equal to 50%, 40%, 30%, 20%, 10%, 5%, 2%, 1.5%, 1%, 0.75%, 0.25%, or 0.1%, wherein the sequence variant does not include a truncation or nucleotide substitution, insertion and/or deletion of the targeting domain and at least about 95%, 96%, 97%, 98%, or 99% of the guide RNA sequences in the composition have a targeting domain identical to the reference guide RNA targeting domain.

In one embodiment, methods disclosed herein can be used to qualify a guide RNA composition for further development (e.g., determine it's suitable for therapeutic development, release into commerce, manufacturing batch release, and the like), wherein at least about 50%, 60%, 70%, 80%, 85%, 90%, 95%, 96%, 97%, 98%, 99% of the guide RNA molecules in the composition are identical to a reference guide RNA. In a particular embodiment, a guide RNA composition is qualified for further development, wherein at least about 95%, 96%, 97%, 98%, 99%, 99.1%, 99.2%, 99.3%, 99.4%, 99.5%, 99.6%, 99.7%, 99.8%, or 99.9% of the guide RNA in 20 the composition are identical to the reference guide RNA.

In one embodiment, methods disclosed herein can be used to disqualify a guide RNA composition from further development (e.g., determine it's not suitable for therapeutic development, release into commerce, manufacturing batch release, and the like), wherein the composition includes:
(a) more than about 95%, 90%, 80%, 70%, 60%, 50%, 40%, 30%, 20%, 10%, 5%, 4%, 3%, 2%, 1%, 0.5%, or 0.1% of truncation variants including a truncation at a 5′ or 3′ end, relative to a reference guide RNA sequence; and/or
(b) more than about 95%, 90%, 80%, 70%, 60%, 50%, 40%, 30%, 20%, 10%, 5%, 4%, 3%, 2%, 1%, 0.5%, 0.3%, or 0.1% of sequence variants including a nucleotide substitution, insertion and/or deletion, relative to a reference guide RNA sequence; and/or
(c) the total amount of guide RNA in the composition is more than about 95%, 90%, 80%, 70%, 60%, 50%, 40%, 30%, 20%, 10%, 5%, 4%, 3%, 2%, 1%, 0.5%, 0.3%, or 0.1% of sequence variants that include a truncation or nucleotide substitution, insertion and/or deletion of the targeting domain relative to the reference guide RNA targeting domain.

In one embodiment, methods disclosed herein can be used to disqualify a guide RNA composition from further development (e.g., determine it's not suitable for therapeutic development, release into commerce, manufacturing batch release, and the like), wherein less than about 95%, 90%, 80%, 70%, 60%, 50% of the guide RNA in the composition do not include a truncation, nucleotide substitution, nucleotide insertion and/or nucleotide deletion relative to a reference guide RNA (e.g., at least about 50%, 60%, 70%, 80%, 85%, 90%, 95%, 96%, 97%, 98%, 99%, 99.1%, 99.2%, 99.3%, 99.4%, 99.5%, 99.6%, 99.7%, 99.8%, or 99.9% of the guide RNA molecules in the composition have the exact sequence as the reference guide RNA). In a particular embodiment, a guide RNA composition is disqualified from further development, wherein at least about 20%, 15%, 10%, 5%, 4%, 3%, 2%, 1%, 0.5%, 0.3%, or 0.1% of the total amount of guide 15 RNA in the composition include a truncation, nucleotide substitution, nucleotide insertion and/or nucleotide deletion relative to the reference guide RNA (e.g., at least about 80% or more of the guide RNA molecules in the composition have the exact sequence as the reference guide RNA and at least about 20% or more of the guide RNA molecules in the composition have a different sequence).

In certain embodiments, methods disclosed herein can be used to determine the identity of a composition of guide RNAs relative to the purity of the composition with respect to the percentage of guide RNA sequences present that match a reference guide RNA sequence. Preparations or samples of guide RNA compositions can be tested for identity using methods disclosed herein for submissions to regulatory authorities before, during, or after clinical trials.

The identity of a guide RNA composition helps determine the quality of the composition or preparation. The quality can be based on predetermined criteria such as the criteria provided herein for high purity, or based on qualifying criteria predetermined, for example, by a regulatory authority. Accordingly, methods and criteria disclosed herein can be used to evaluate preparations of guide RNAs before or during a manufacturing process. For example, preparations can be tested using methods disclosed herein to determine the quality of a guide RNA product being manufactured as a therapeutic or clinical therapeutic candidate.

Methods disclosed herein can be used to qualify manufacturing of GMP (Good Manufacturing Practice) guide RNA compositions, e.g., to determine if the composition has sufficient purity as described herein for development purposes (e.g., suitable for therapeutic development, release into commerce, manufacturing batch release, and the like). Methods disclosed herein can be used to disqualify manufacturing of GMP (Good Manufacturing Practice) guide RNA compositions, e.g., to determine if the composition does not have sufficient purity for development purposes as described herein.

As used herein, the term “preparation” can be, for example, a sample, batch, and/or lot including guide RNAs of interest that is produced, for example, during a manufacturing process or for a proposed manufacturing process or taken from a batch or lot approved for clinical or commercial purposes (e.g., to test the quality for safety monitoring).

Methods disclosed herein can be used for any purpose where quality control is needed before, during, and after manufacturing of guide RNA preparations and compositions. For example, disclosed methods can be used in determining release of a manufactured batch into commerce or to qualify a manufacturing process for a particular guide RNA drug substance.

In certain embodiments, methods disclosed herein can be used to complement or confirm quality tests performed using other methods of quality control for RNA molecule preparations. Such other methods include, but are not limited to, ESI (electrospray ionization) and HPLC (high-performance liquid chromatography) methods used to determine the purity (e.g., the percentage of RNA molecule variants in a preparation).

Methods disclosed herein can be used to compare the quality of different preparations of RNA (e.g., guide RNA) to each other. For example, a sample can be taken from a preclinical batch and compared with a batch manufactured during a clinical trial or intended for commercial launch or for validating a process for GMP purposes. Thus, the methods can be used to ensure consistent quality in a manufacturing process used to make RNA molecules (such as guide RNA) at different times.

Results provided by methods disclosed herein can be reported as an assessment and included, for example, in a print or computer-based form. For example, as a batch or lot record, a report, Material Safety Data Sheet, Certificate of Testing, Certificate of Analysis.

### Sequencing

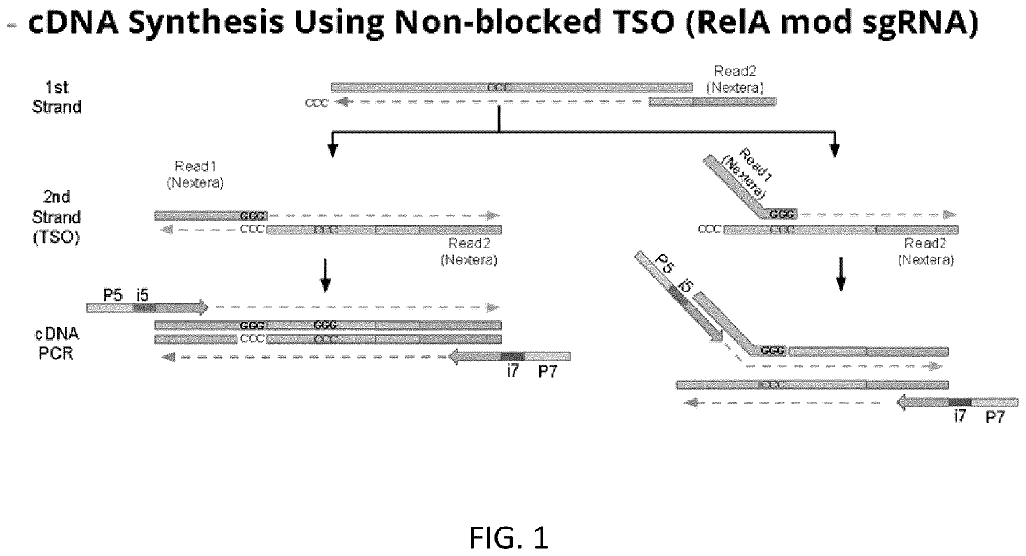

Methods for assessing a guide RNA composition are provided, including determining the sequence of the guide RNA molecules in the composition. Various sequencing methods to determine the sequence of RNA molecules are known in the art and can be adapted for use according to the present disclosure. In certain embodiments, a template switching oligonucleotide (TSO) is used in a method of the disclosure. In certain embodiments, a primer is designed to hybridize to the 3′ end of the guide RNA that can be extended by a reverse transcriptase to form a first DNA strand that uses the guide RNA as a template. When reaching the 5′ end of the guide RNA the reverse transcriptase can add additional nucleotides to the cDNA strand that overhang the template guide RNA sequence, such as a tri-cytosine repeat sequence (3′-CCC-). A TSO can be used that has a 3′ tri-guanine repeat sequence (-GGG-3′) that hybridizes to the 3′-CCC- sequence at the end of the first strand. Polymerization can be initiated to form a second strand extending the TSO in the 3′ direction. FIG. 1 shows this approach. The problem with this approach, as shown in FIG. 1, is the generation of less than full-length cDNA versions of the template guide RNA where the TSO hybridizes to a tri-cytosine sequence that happens to be internal to the guide RNA (i.e., not at the 3′ tri-cytosine repeat added by the reverse transcriptase during first strand extension). A TSO can also include a ribonucleotide at the 3′ end to hybridize to the tri-nucleotide repeat left over from first strand synthesis.

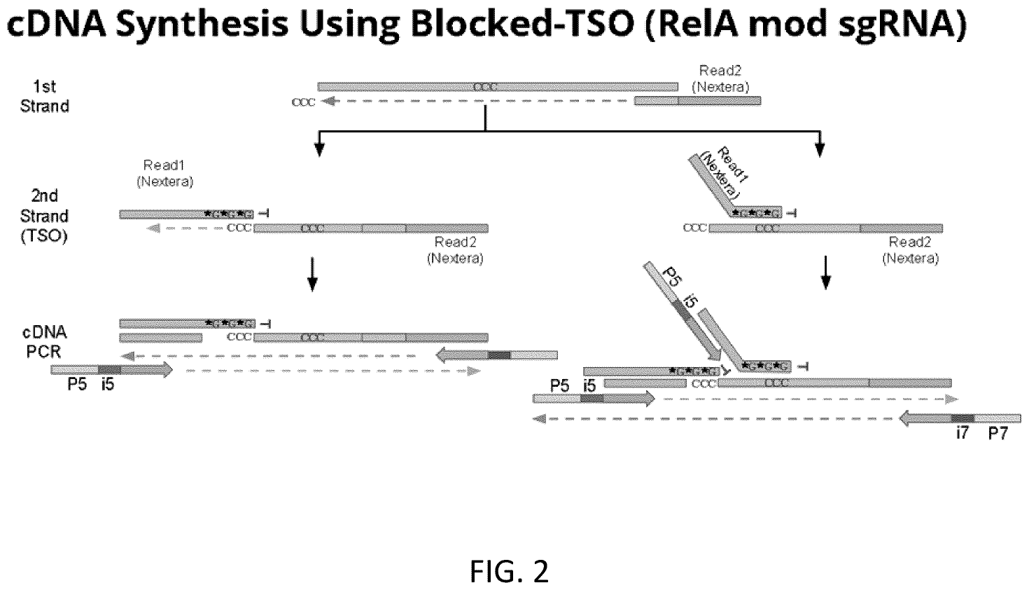

As shown in FIG. 2, this problem can be solved by blocking the 3′ end of the TSO such that a second strand is not formed when it hybridizes to an internally located tri-cytosine repeat sequence. Accordingly, TSO are disclosed herein including a modification at the 3′ end that can block DNA polymerization during cDNA synthesis. In certain embodiments, the 3′ TSO modification is a 3-Dideoxy Cytosine (3ddC) modification located at the 3′ end of the TSO. In certain embodiments, a TSO used in a disclosed method includes three riboguanines at the 3′ end to which the 3ddC is attached.

Sequencing platforms useful in the methods disclosed herein are well known in the art, including but not limited to those provided by Illumina, Thermo Fisher Scientific (ION TORRENT®), GenapSys, Qiagen, and BGI-Shenzhen.

In certain embodiments, sequence adapters can be included in the primer and/or the TSO. Typically, the adapters are included at the 5′ ends of the primer and/or TSO. Where adapter(s) are provided, a cDNA strand generated during DNA polymerization can include an adapter sequence at the 5′ end from the primer and the 3′ end from the TSO.

In some embodiments, a guide RNA preparation for cDNA synthesis includes the guide RNA, which serves as the template for first strand synthesis, in an amount suitable for a reaction to occur that generates cDNA copy of the guide RNA. The final amount of guide RNA in the preparation can be about 500 pg to about 1 μg.

In certain embodiments, the guide RNA can include additional nucleotide(s) at the 5′ end or 3′ end. For example, the guide RNA can include a polyA tail (e.g., adenine residues can be added using adenylation methods well known to those of skill in the art, for example using a Poly(A) polymerase).

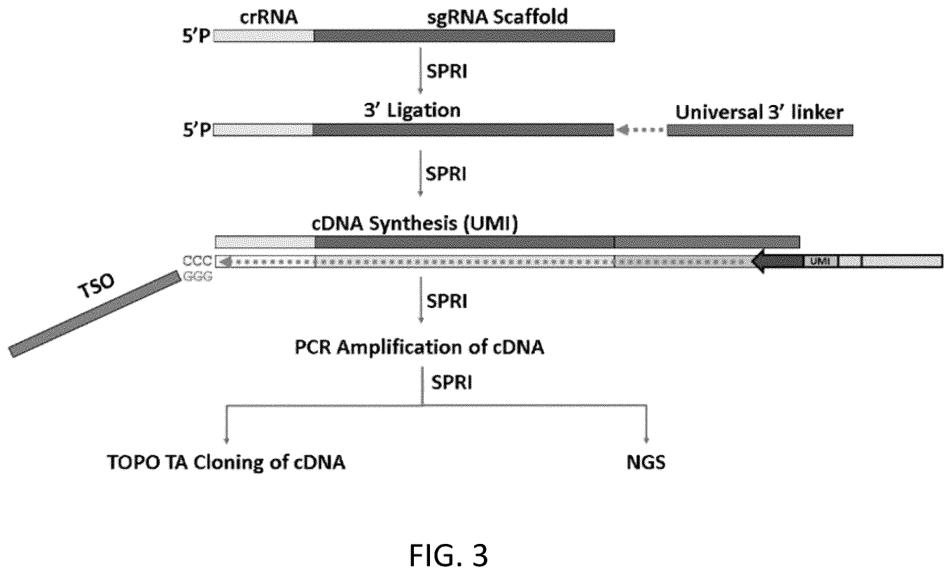

In a particular embodiment, an adapter molecule (also referred to herein as a linker or universal linker) can be ligated to the 3′ end of the guide RNA (see FIG. 3). Such adapters can be designed by one of skill in the art or obtained from a commercial vendor, such as IDT or Illumina, Inc. The adapter molecule can contain an adapter sequence that provides a priming site for a primer to begin the first strand synthesis in a reverse transcription process.

In some embodiments a linker or an adapter molecule can obtain a UMI that can help to determine sequencing error correction and accuracy. UMIs are also well described in the literature and available by various commercial vendors.

In certain embodiments, methods disclosed herein include a polymerase that can extend a first strand of cDNA and then continue extension of a second strand from the 3′ end of a TSO. Suitable polymerases include reverse transcriptases (RT), including but not limited to Maxima H-minus RT (Thermo Fisher Scientific, Waltham, MA), SuperScript IV (Thermo Fisher Scientific, Waltham, MA), SMART® MMLV Reverse Transcriptase (Takara Bio, San Jose, CA). In certain embodiments, the final concentration of polymerase (e.g., RT) in the reaction preparation is about 1 unit to about 20 units where one unit can process 1 nmol or an equivalent amount of RNA template into cDNA. The use of TSOs for generating cDNAs is described, for example, in Zhu et al., 2001, Biotechniques, 30(4):892-897 and Kapteyn et al., 2010, BMC Genomics, 11:413.

In certain embodiments, the polymerase can add nucleotides (e.g., deoxyribonucleotides) at the 3′ end of the cDNA molecule being generated during a method disclosed herein. Thus, the first strand cDNA copy of the guide RNA template strand will have additional nucleotides at the 3′ end, such as 1, 2, 3, 4, 5, or more additional nucleotides at the 3′ end beyond the 5′ end of the template guide RNA sequence. In some embodiments, the polymerase adds about 3 deoxynucleotides to the 3′ end of the first strand cDNA. In a particular embodiment, the polymerase adds 3 cytosines to the 3′ end of the first strand cDNA.

In certain embodiments, the reaction preparation in disclosed methods includes a TSO as described herein at a concentration suitable for allowing the polymerase to generate a second strand cDNA after the first strand is generated. The TSO can be added, for example, from a stock solution having a concentration of about 100 uM and added to the mixture for second strand synthesis at a final concentration of about 0.1 μM to about 10 μM, for example 0.1 μM).

The TSO can be, for example, about 30-36 nucleotides in length. A sequence is provided in the 3′end of the TSO that can hybridize to the first strand cDNA thereby priming it for second strand synthesis. In one embodiment, the hybridizing domain is in the 3′ end of the TSO as described herein. TSOs are described, for example, in U.S. Pat. No. 5,962,272. TSOs are also commercially available, for example from Takara Bio (San Jose, CA).

A TSO provided herein can also include an adapter sequence, such as is used by a sequencing platform known to those of skill in the art. For example, the sequence adapter can bind to a surface-bound oligonucleotide on a flow cell (e.g., a p5 and p7 oligonucleotides used in the Illumina NGS sequencing platforms). Adapters specific for various sequencing platforms are commercially available and described in detail by the manufacturers and distributors on their websites and in their literature. Those of skill in the art can readily ascertain appropriate adapter sequences. Sequence adapters can be, for example 1 μM to about 200 μM, any length as selected by one of skill in the art depending on the sequencing platform appropriate being used.

The TSO can also include a unique molecular identifier (UMI) as known in the art to help determine sequencing error correction and accuracy. UMIs are also well described in the literature and available by various commercial vendors.

In some embodiments, a TSO may include: a dideoxycytosine at the 3′ terminus, three guanosines in the 3′ end, and a sequence adapter for sequencing. Optionally, the TSO may include a UMI.

In certain embodiments, the disclosed methods include dNTPs added to reaction preparation including the other components described herein to enable synthesis of cDNA. As used herein, “dNTPs” includes all of the deoxynucleotides (dATP, dTTP, dGTP, and dCTP). These can be added to a first strand mixture or a second strand mixture, or a mixture including all of the components for generating the first and second strands and for carrying out the methods disclosed herein. The dNTPs can be added to a reaction preparation so that the final concentration is about 0.1 mM to about 0.5 mM. In one embodiment, the final concentration is about 0.375%.

In certain embodiments, methods disclosed herein include adding primers that hybridize to the guide RNA or to an adapter ligated to the guide RNA for synthesis of a first strand DNA. This step is conducted under conditions suitable for hybridization of the primer to the intended target sequence. The primer can be designed to specifically hybridize to a complementary sequence of the guide RNA sequence itself, of the adapter sequence ligated to the guide RNA, or of both the guide RNA and ligated adapter. The primer may also include a sequence that does not hybridize to the guide RNA or a ligated adapter. In this case, the 5′ end portion of the primer will overhang while its 3′ end portion hybridizes to a target sequence on the guide RNA, ligated adapter, or both. The non-hybridizing portion of a primer can include a sequence adapter specific for a sequencing platform as described herein. It can also include a UMI as described herein.

One skilled in the art will readily recognize that the primer can be any length suitable for the intended purpose. Primer stock concentrations can range from 1 μM to 100 μM and final concentrations can be from 0.1 μM to 2 μM or as needed in the assay.

### UMI-Based Quantification

Unique Molecular Identifier (UMI) can be incorporated to correct bias in library preparation and sequencing processes and to allow quantitative evaluation of RNA samples. The UMI sequences can be added during the ligation step to the 3′-end of analyzed RNA molecules and/or the reverse transcription step when applicable to a method disclosed herein. To achieve the goal of UMI-based quantification, it is important that the combination of UMI sequences is large enough to cover the number of molecules in the analyzed RNA sample. For example, UMI sequences of 10-nucleotide (10-nt) length have 4¹⁰ (˜1 million) different combinations. However, such UMI design would not be sufficient for an RNA sample of 3 pmol, which contains about 1.8 trillion molecules (e.g., 1.8 million-fold more than the UMI combinations). Such phenomenon, where there are not enough UMI combinations to cover every molecule in a sample and thus multiple molecules will be tagged with the same UMI sequence, is usually referred to as UMI collision. In the earlier example with 3 pmol of RNA sample, to guarantee that each molecule is tagged with a unique UMI sequence, the UMI sequence need to be at least 21-nt long based on log₄ 1.8×10¹²≈20.4.

Another factor to consider in the application of UMI is the number of reads collected on the sequencing machine. For example, an Illumina iSeq 100 instrument can produce ˜4 million reads per run, which are shared across indexed samples. For example, if 32 samples are indexed and run together on an iSeq instrument, each sample will have on average at most 125,000 reads. If again 10-nt UMI sequences are used with these 32 samples, the UMI collision observed in the final sequencing results will be much mild, because statistically only a small proportion (˜12%) of the UMI combinations are examined.

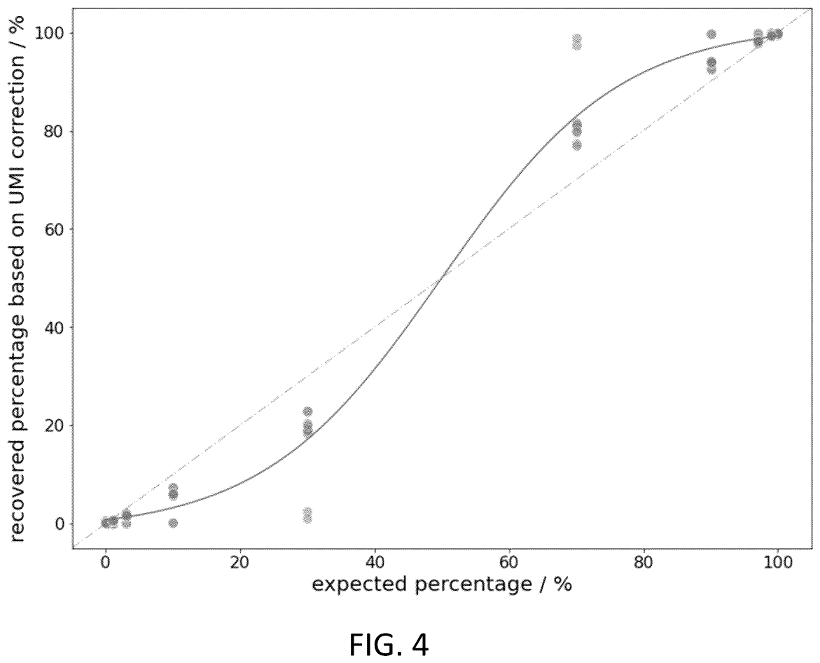

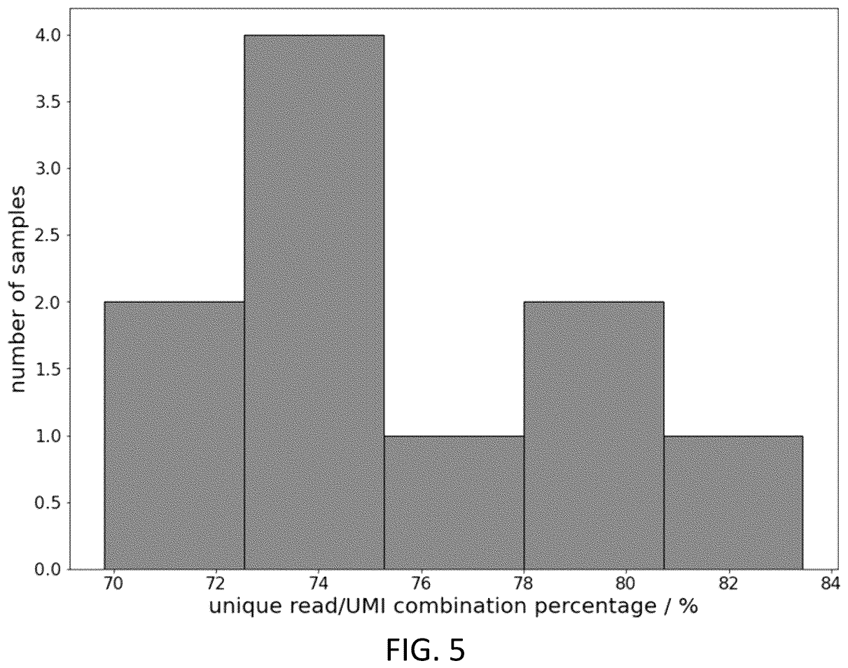

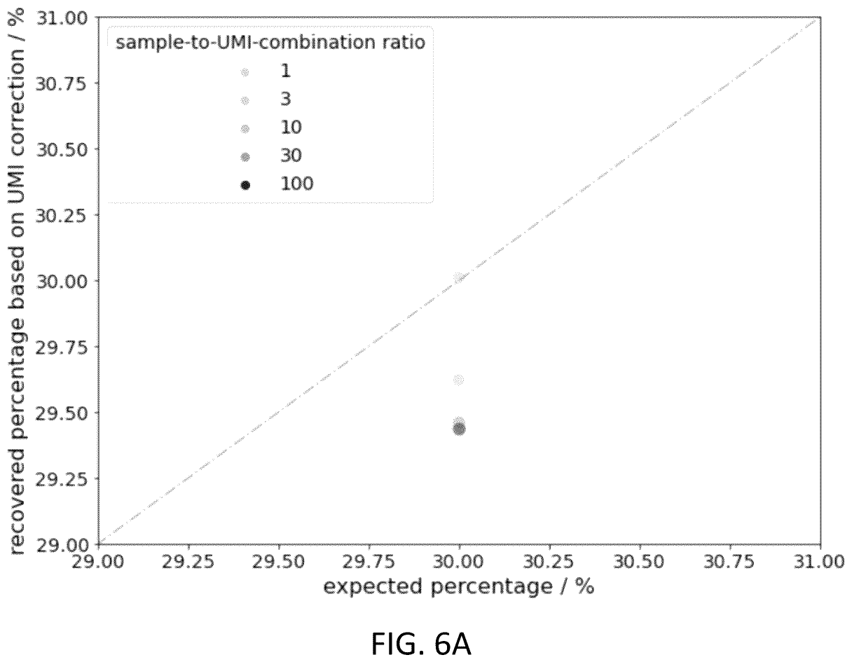

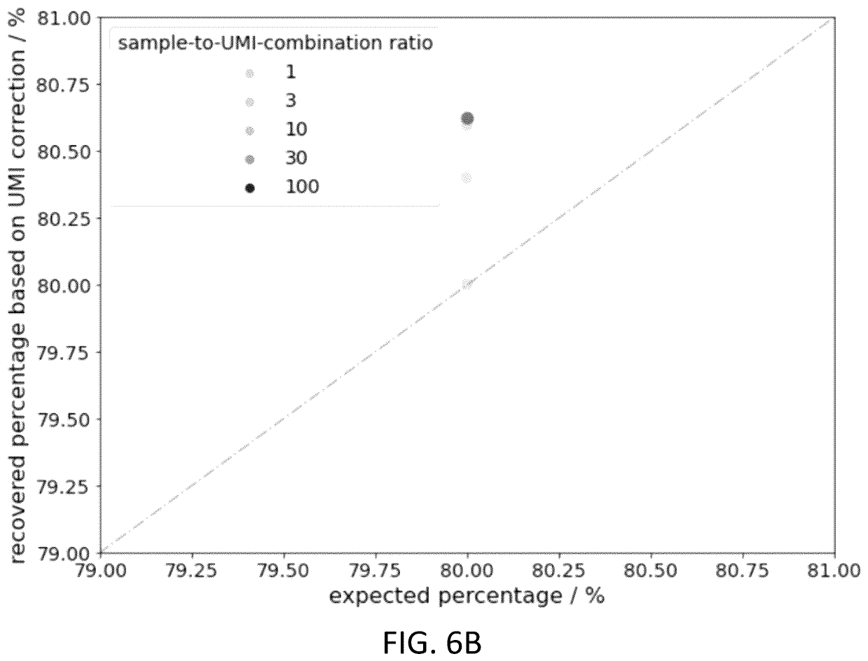

The aforementioned two factors, the sample-to-UMI-combination ratio and the read number of sequencing were examined in both wet-lab experiments and dry-lab (e.g., in silico) simulation. For wet-lab experiments, individual RelA single-nucleotide variant (SNV) and RelA were mixed with wildtype RelA at a constant total yield of 3 pmol but different molar percentages, namely, 0.1%, 1%, 3%, 10%, 30%, and 100%. UMI sequences of 10-nt length were added during the ligation step to the RNA 3′-end. The mixture RNA samples were then converted to cDNAs which were further amplified by PCR. The PCR products were sequenced on an Illumina iSeq 100 instrument. Shown in FIG. 4 is the scatterplot of recovered percentages of SNV and RelA species based on UMI correction against their expected percentages. If the sequencing method is not affected by UMI collision, the recovered percentages should match the expected values and all data points should lie right on the y=x diagonal line. However, the observation in FIG. 4 was that the percentage of the minority species (e.g., data point with expected percentages <50%) in a mixture was consistently underestimated after the UMI correction, while that of the majority species (e.g., data point with expected percentages >50%) was always overestimated. Such behavior is consistent with the simulated UMI collision discussed later (FIG. 6A and FIG. 6B). Another metric to examine the UMI collision is the percentage of UMI sequence with unique RNA sequence read over all UMI sequences found in the sequencing results, i.e., unique read/UMI combination percentage. If there is no UMI collision in the sequencing experiment, the unique read/UMI combination percentage should be close to 100% (e.g., ≥99%) with minor deviations caused by inherit reading error rates on the sequencer. Plotted in FIG. 5 is the distribution of unique read/UMI combination percentages in all the 30% molar percentage samples. Consistent with the sigmoid shape observed in FIG. 4, the unique read/UMI combination percentages also suggested that 10-nt long UMI sequences were not sufficient for 3 pmol of RNA sample. However, the unique read/UMI combination percentages would've been much lower (1%), if the number of reads was equal or larger than the UMI combinations (i.e., ≥1 million reads). In fact, with 10-nt long UMI, our in silico simulation of an RNA sample in just 2× excess to UMI combinations (i.e., 2 million molecules, ˜3 attomole) showed that the unique read/UMI combination percentages would be 58.02±0.06% (n=5). In the wet-lab, the number of molecules in an RNA sample is a million-fold excess to UMI combinations. Such dramatic difference showed the impact of read numbers in scenarios of UMI collision.

The impact of the sample-to-UMI-combination ratio on UMI correction was further investigated with in silico simulation. In the simulation study, the UMI length was kept as 10 nucleotides, and the number of reads was kept at 140,000. Due to the limit in computational resources, the sample-to-UMI-combination ratios examined were 1, 3, 10, 30, and 100. Because the ratio values were relatively small compared to those in wet-lab experiments (e.g., 1 million), their impacts on the UMI correction were also less significant, which is consistently with our observation about the impact of read numbers on UMI collision. However, when compared across the different ratio values, the trend was very clear. FIG. 6A and FIG. 6B showed the recovered percentages based on UMI correction in samples with expected percentages of 30% and 80%, respectively. Similar to the observation in wet-lab experiments (FIG. 4), underestimation of species percentages was observed in the 30% samples, while overestimation was observed in the 80% samples. More importantly, there was clearly this trend that the larger the sample-to-UMI-combination ratio, the larger the degree of underestimation or overestimation. Such trend played a crucial role in reassuring that UMI collision can be avoided if we reduce the sample-to-UMI-combination ratio close to 1. To achieve such reduction in wet-lab experiments, it would be more practical to increase the length of UMI sequences than to significantly reduce the sample size. Therefore, the exploration of suitable UMI sequence length is an important process in the sequencing methods disclosed here.

### Compositions and Kits

Disclosed herein are compositions including a template switching oligonucleotide (TSO) that has a 3′end modification that prevents DNA polymerization. In certain embodiments, the modification is a dideoxynucleotide (e.g., ddC, ddG, ddT, or ddA). In a particular embodiment, the modification is a ddC. In another embodiment, the TS) has a tri-nucleotide repeat sequence at the 3′ end to which the 3′ end modification is attached, for example, the TSO has three guanines at the 3′ end to which a dideoxynucleotide is attached (e.g., -GGG-ddC-3′). In some embodiments, the three nucleotides at the 3′ end of the TSO are ribonucleotides or ribonucleosides or a combination thereof. For example, the 3′ end of the TSO can be -rGrGrG-ddC-3′, where rG is guanosine.

Also, disclosed herein are kits including compositions provided herein as well as one or more reagents or other such components needed to generate a cDNA library based on a guide RNA template and perform NGS or any other type of sequencing to determine the sequence of guide RNAs in a preparation of guide RNAs.

### Exemplary Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure numbered 1-37 are provided below. As will be apparent to those of skill in the art upon reading this disclosure, each of the individually numbered aspects may be used or combined with any of the preceding or following individually numbered aspects. This is intended to provide support for all such combination of aspects and is not limited to combinations of aspects explicitly provided below.

### EXEMPLARY EMBODIMENTS

1. A method for determining a sequence of a RNA, comprising:
   a. providing a RNA from a sample;
   b. preparing a first and second strand mix comprising:
      i. the RNA;
      ii. DNA primer that hybridizes to the 3′ end of the RNA;
      iii. dNTPs;
      iv. a template switching oligonucleotide (TSO) comprising a modification at the 3′ terminal end; and
   c. incubating the first strand mix and the second strand mix under conditions to allow synthesis of cDNA;
   d. optionally, purifying the cDNA;
   e. amplifying the cDNA;
   f. optionally, purifying the amplified cDNA; and
   g. sequencing the amplified cDNA, wherein the RNA is optionally a guide RNA.
2. A method for determining a sequence of a RNA, universal adapter oligonucleotide comprising:
   a. providing a RNA from a sample;
   b. preparing a ligation mix comprising the RNA, a universal adapter oligonucleotide containing a priming sequence that is ligated to the 3′ end of the RNA molecule; RNA ligase; and a template switching oligonucleotide (TSO) comprising a modification at the 3′ terminal end;
   c. incubating the ligation mix under conditions to allow ligation of the DNA oligo to the 3′ end of the RNA;
   d. purifying the ligated RNA;
   e. preparing a first and second strand mix comprising: the ligated RNA, DNA primer that hybridizes to the priming sequence in the ligated universal adapter oligonucleotide, dNTPs, and a template switching oligonucleotide (TSO) comprising a modification at the 3′ terminal end; and
   f. incubating the first strand mix and the second strand mix under conditions to allow synthesis of cDNA;
   g. optionally purifying the cDNA;
   h. amplifying the cDNA;
   i. purifying the amplified cDNA; and
   j. sequencing the amplified cDNA, wherein the RNA is a guide RNA.
3. The method of embodiment 1 or 2, wherein the modification is a 3-dideoxycytosine (3ddC).
4. The method of any one of the preceding embodiments, further comprising comparing the sequences with a reference guide RNA sequence.
5. The method of any one of the preceding embodiments, wherein the cDNA is amplified by PCR.
6. The method of any one of the preceding embodiments, wherein the sequencing comprises NGS.
7. The method of any one of the preceding embodiments, wherein the amplified cDNA is cloned into expression vectors for Sanger sequencing.
8. The method of any one of the preceding embodiments, wherein the RNA is about 40 nucleotides to about 120 nucleotides in length and the RNA is optionally a guide RNA.
9. The method of any one of the preceding embodiments, wherein a sequence is determined for the entire RNA and the RNA is optionally a guide RNA.
10. The method of any one of the preceding embodiments, wherein the 3′ end of the TSO comprises the sequence -GGG-3ddC or -rGrGrG-3ddC (i.e., the modification is at the 3′ end).
11. The method of any one of the preceding embodiments, wherein the 3′ end of the TSO comprises the sequence -GGGG-3ddC or -GrGrGrG-3ddC (i.e., the modification is at the 3′ end).
12. A method for preparing a cDNA copy of a guide RNA, comprising:
   a. providing a guide RNA from a sample;
   b. preparing a first and second strand mix comprising:
      i. the guide RNA
      ii. DNA primer that hybridizes to the 3′ end of the guide RNA
      iii. dNTPs
      iv. a template switching oligonucleotide (TSO) comprising a modification at the 3′ end, and
      v. a reverse transcriptase; and
   c. incubating the first strand mix and the second strand mix under conditions to allow synthesis of cDNA.
13. The method of embodiment 12, wherein the guide RNA is 100mer.
14. The method of embodiments 11-13, wherein a sequence is determined for the entire guide RNA.
15. The method of embodiments 11-14, wherein the modification is a 3-dideoxyc(3ddC).
16. The method of embodiments 11-15, wherein the 3′ end of the TSO comprises the sequence -GGG-3ddC or -rGrGrG-3ddC (i.e., the modification is at the 3′ end).
17. The method of embodiments 11-16, wherein the 3′ end of the TSO comprises the sequence -GGGG-3ddC or -GrGrGrG-3ddC (i.e., the modification is at the 3′ end).
18. A template switching oligonucleotide (TSO) comprising a modification at the 3′ end that 15 inhibits DNA polymerase activity.
19. The TSO of embodiment 18, wherein the modification is a 3-dideoxycytosine (3ddC).
20. The TSO of embodiment 19, wherein the 3′ end of the TSO comprises the sequence -GGG-3ddC or -rGrGrG-3ddC (i.e., the modification is at the 3′ end).
21. The TSO of embodiment 19, wherein the 3′ end of the TSO comprises the sequence -GGGG-3ddC or -GrGrGrG-3ddC (i.e., the modification is at the 3′ end).
22. A cDNA library preparation mixture, comprising:
   a. a guide RNA;
   b. DNA primer that hybridizes to the 3′ end of the guide RNA;
   c. a template switching oligonucleotide (TSO) comprising a modification at the 3′ end (e.g., 3ddC);
   d. optionally dNTPs; and
   e. optionally a reverse transcriptase.
23. A kit for generating cDNA copies of a guide RNA, comprising a template switching oligonucleotide (TSO) that has a 3-Dideoxy Cytosine (3ddC) modification at the 3′ end.
24. The kit of embodiment 23, further comprising a DNA primer that hybridizes to the 3′ end of a guide RNA.
25. The kit of embodiment 23, further comprising dNTPs.
26. The kit of embodiment 23, further comprising a reverse transcriptase.
27. A method for assessing the identity of a guide RNA in a sample, comprising:
   a. providing a guide RNA from the sample;
   b. preparing a first and second strand mix comprising:
      i. the guide RNA
      ii. DNA primer that hybridizes to the 3′ end of the guide RNA
      iii. dNTPs
      iv. a template switching oligonucleotide (TSO) comprising a modification at the 3′end
   c. incubating the first strand mix and the second strand mix under conditions to allow synthesis of cDNA,
   d. sequencing the amplified cDNA
   h. comparing the sequence of the amplified cDNA to a reference guide RNA sequence.
28. The method of embodiment 27, wherein the modification is a 3-dideoxycytosine (3ddC).
29. The method of any one of embodiments 27-28, further comprising the step of ligating a universal adapter oligonucleotide to the guide RNA between steps a) and b).
30. The method of any one of embodiments 27-29, further comprising comparing the sequences with a reference guide RNA sequence.
31. The method of any one of embodiments 27-30, wherein the cDNA is amplified by PCR.
32. The method of any one of embodiments 27-31, wherein the sequencing comprises NGS.
33. The method of any one of embodiments 27-32, wherein the amplified cDNA is cloned into expression vectors for Sanger sequencing.
34 The method of any one of embodiments 27-33, wherein the guide RNA is about 40 to about 120 nucleotides in length.
35. The method of any one of embodiments 27-34, wherein a sequence is determined for the entire guide RNA (e.g., not the targeting sequence only).
36. The method of any one of embodiments 27-35, wherein the 3′ end of the TSO comprises the sequence -GGG-3ddC or -rGrGrG-3ddC (i.e., the modification is at the 3′ end).
37. The method of any one of embodiments 27-36, wherein the 3′ end of the TSO comprises the sequence -GGGG-3ddC or -GrGrGrG-3ddC (i.e., the modification is at the 3′ end).
38. A method for determining a sequence of an RNA molecule, comprising:
   a. providing a guide RNA from a sample;
   b. preparing a first and second strand mix comprising:
      i. the RNA molecule;
      ii. DNA primer that hybridizes to the 3′ end of the RNA molecule;
      iii. dNTPs;
      iv. a template switching oligonucleotide (TSO) comprising a modification at the 3′ terminal end; and
   c. incubating the first strand mix and the second strand mix under conditions to allow synthesis of cDNA;
   d. sequencing the cDNA.
39. A method for determining a sequence of an RNA molecule, comprising:
   a. providing a guide RNA from a sample;
   b. preparing a ligation mix comprising the RNA molecule, an adapter oligonucleotide containing adapter sequence that is covalently ligated to the 3′ end of the RNA molecule; RNA ligase; and a template switching oligonucleotide (TSO) comprising a modification at the 3′ terminal end;
   c. incubating the ligation mix under conditions to allow ligation of the DNA oligonucleotide to the 3′ end of the RNA molecule;
   d. purifying the ligated RNA molecule;
   e. preparing a first and second strand mix comprising: the ligated RNA molecule, DNA primer that hybridizes to the adapter sequence in the ligated DNA oligonucleotide, dNTPs, and a template switching oligonucleotide (TSO) comprising a modification at the 3′ terminal end; and
   f. incubating the first strand mix and the second strand mix under conditions to allow synthesis of cDNA; and
   g. sequencing the cDNA.
40. The method of embodiment 38 or 39, wherein the modification is a 3-dideoxycytosine (3ddC).
41. The method of embodiment 38 or 39, wherein the RNA molecule is a guide RNA.
42. The method of any one of the preceding embodiments, wherein purification between steps is not carried out.

### EXAMPLES

The following examples are illustrative and should not be interpreted to limit the scope of the claimed subject matter.

### Example 1

### Use of Template Switching Oligonucleotides for Guide RNA Sequencing

A method for single guide RNA (sgRNA) sequence verification was developed based on cDNA synthesis from the guide RNA using a primer specific for the 3′ end of the sgRNA for a first strand synthesis. A reverse transcriptase adds 3 cytosines at the end of the first strand synthesis. The second strand synthesis is started using a template switching oligo (TSO), that contains 3 Guanines at the 3′ end of the TSO that will prime at the 3 cytosines introduced by the reverse transcriptase. This method was designated “TSO v1.” The sequence of the TSO used in TSO v1 was TCGTCGGCAGCGTCAGATGTGTATAAGAGACAGrGrGrG (SEQ ID NO: 6).

### Limitation of the TSO v1 Method

One major limitation of the TSO v1 method is that if the sgRNA sequence contains a homopolymer of ≥3 Guanines anywhere in its sequence, the TSO can potentially prime both at the 3 cytosines introduced by the reverse transcriptase at the end of the first strand, but also to any homopolymer of ≥3 guanine (G) residues, which are always located before the 3 cytosine nucleotides introduced by the reverse transcriptase.

When that happens, the mis-priming of TSO in a homopolymer of ≥3 Gs, creates artifactual short cDNA molecules (see FIG. 1) that are amplified during the indexing PCR with a bias towards the mis-primed cDNA molecules.

### Example 2

Use of Template Switching Oligonucleotides with 3′ Modifications to Improve Guide RNA Sequencing Results

To bypass the mis-priming of TSO in homopolymers of ≥3 Glys, chemical modifications were added at the 3′ end of the TSO, including substitution of the 3′OH by (1) 3 phosphorothioate moieties, (2) a 3 carbon spacer (3C) or (3) a 3 dideoxycytosine (3ddC). Those modifications won't avoid the mis-priming during cDNA but would avoid the extension of mis-primed, modified TSO molecules by DNA polymerases during indexing PCR, especially HiFi DNA polymerases (FIG. 2).

HPLC-purified and modified RelA single guide RNAs (“sgRNAs”) were used. The RelA sgRNA contained a homopolymer of >3 Gs in its crRNA sequence and was observed previously to produce artifacts and short cDNA molecules during a cDNA synthesis protocol that used a template switch oligonucleotide (TSO) that had 3 cytosines at its 3′ end for priming.

Three moieties of phosphorothioate (PTA), which were shown previously not to block DNA polymerases during indexing PCR of 100-mer with 3 Gs, were used as a control. The TSO-PTA vs TSO-3C vs TSO-3ddC were compared at two different concentrations in the following 3×3 matrix:

TABLE 2
Example matrix to compare TSO-PTA, TSO-
3C, and TSO-3ddC concentrations.

| | | |
|---|---|---|
| 2.5 μM no UMI | 2.5 μM with UMI | 10 μM with UMI |

TSO-PTA
TSO-3C
TSO-3ddC

cDNA Synthesis

First strand cDNA mix was prepared on ice including 0.125 μL of Recombinant RNase Inhibitor (Takara Bio, USA, San Jose, CA) 0.375 μL of 10 mM dNTPs (Takara), 0.1 μL of 100 μM primer specific oligonucleotide (Integrated DNA Technologies, Inc., Coralville, Iowa):

(SEQ ID NO: 7)
5′-TCTCGTGGGCTCGGAGATGTGTATAAGAGACAGaaaagcaccgact
cggt-3′,

0.9375 μL of 40% PEG (Milipore Sigma) and 4.66 μL of nuclease-free water.

The second strand mix was prepared with 10 μL 100 mM Tris-HCL pH 8.3, 0.75 μL 100 mM NaCl, 0.25 μL 10 mM GTP, 0.625 mM MgCl₂, 0.2 μL 100 mM DTT, 1 μL 100 μM template switch oligonucleotide (TSO), and 0.25 Maxima H-minus RT (Thermo Fisher Scientific, Waltham, MA).

Three first strand cDNA/TSO mixes were made with three different TSOs. Each of the TSOs were independently mixed with 6.2 μL of first strand mix was added to 100 ng of sgRNA in 1 μL. The TSOs were:

1)
(SEQ ID NO: 8)
TCGTCGGCAGCGTCAGATGTGTATAAGAGACAGrGrGrG-PTA
2)
(SEQ ID NO: 9)
TCGTCGGCAGCGTCAGATGTGTATAAGAGACAGrGrGrG-3C
3)
(SEQ ID NO: 10)
TCGTCGGCAGCGTCAGATGTGTATAAGAGACAGrGrGrG-3ddC

Each first strand cDNA/TSO mix was incubated for 5 minutes at 70° C. then snap cooled on ice to denature the sgRNA secondary structure.

Afterward, 2.8 uL of second Strand Mix was added to the snap cooled mixture then incubated using the following protocol to synthesize first and second cDNA strands.

TABLE 3
Example protocol to synthesize first and
second cDNA strands from Example 2.

| Step # | Temp (° C.) | Time in min. | # Cycles |
|---|---|---|---|
| 1 | 42° C. | 90 min. | 1 |
| 2 | 50° C. | 2 min. | 10 |
| | 42° C. | 2 min. | |
| 3 | 85° C. | 5 min. | 1 |
| 4 | 12° C. | on hold | 1 |

cDNA product was purified with SPRI (MagBio Genomics, Gaithersburg, MD) beads at a 2.5×ratio eluted in 20 μL 1×TE buffer, pH 8.0.

A PCR master mix was prepared using 5 μL 5×GXL Buffer (Takara), 2.5 μL dNTPs (Takara), 0.5 μL GXL Polymerase (Takara) and 13.5 μL molecular biology grade H₂O per reaction. 1 μL 2.5 μM Primer 1, composed of P5 adapter sequence (required to anneal to its complementary oligo on the Illumina flow cell for sequencing, followed by i5 index sequence (sequence identifier) and 5′ universal linker (illumina sequencing primer binding site), 1 μL 2.5 μM Primer 2, composed of P7 adapter sequence (required to anneal to its complementary oligo on the Illumina flow cell for sequencing), followed by i7 index sequence (sequence identifier) and 3′ universal linker (Illumina sequencing primer binding site) and 2 μL purified cDNA were added to the reaction. The PCR was cycled using the following program.

TABLE 4
Example PCR protocol used in Example 2.

| Step | Temp | Duration | Cycles |
|---|---|---|---|
| 1 | 98° C. | 30 sec | 1X |
| 2 | 98° C. | 10 sec | 20X |
| | 60° C. | 15 sec | |
| | 65° C. | 30 sec | |
| 3 | 65° C. | 2 min | 1X |
| 4 | 4° C. | hold | 1X |

PCR products were purified using a 1.6× ratio of SPRI and eluted in 20 uL. PCR products were quantified by Qubit HS dsDNA Kit (Thermo Fisher) prior to loading it in the HS dsDNA Bioanalyzer chip (Agilent). Samples were sequenced using an iSeq 2×150 kit at 60 pM concentration.

cDNA product was purified with SPRI (MagBio Genomics, Gaithersburg, MD) beads at a 2.5× ratio eluted in 20 μL 1×TE buffer, pH 8.0. A PCR master mix was prepared using 5 μL 5×GXL Buffer (Takara), 2.5 μL dNTPs (Takara), 0.5 μL GXL Polymerase (Takara) and 13.5 μL molecular biology grade H₂O per reaction. 1 μL 2.5 μM Primer 1, composed of P5 adapter sequence 10 (required to anneal to its complementary oligo on the Illumina flow cell for sequencing, followed by i5 index sequence (sequence identifier) and 5′ universal linker (Illumina sequencing primer binding site), 1 μL 2.5 μM Primer 2, composed of P7 adapter sequence (required to anneal to its complementary oligo on the Illumina flow cell for sequencing), followed by i7 index sequence (sequence identifier) and 3′ universal linker (Illumina sequencing primer binding site) and 2 μL purified cDNA were added to the reaction. The PCR was cycled using the following program.

Raw fastq files were processed using the following steps. Overall high throughput illumina NGS sequencing quality was assessed using FastQC (Andrews, S. (2010). FastQC: A Quality Control Tool for High Throughput Sequence Data [Online]. Available online at: bioinformatics.babraham.ac.uk/projects/fastqc/per fastq file). Trimmomatic (Bolger, A. M., Lohse, M., & Usadel, B. (2014). Trimmomatic: A flexible trimmer for Illumina Sequence Data.) was used to trim the Illumina sequencing adapters from 5′ and 3′ ends of the reads and to filter for quality reads with a Phred Quality score >Q30. Quality filter and adapter trimmed paired reads were then merged using flash (FLASH: Fast length adjustment of short reads to improve genome assemblies. T. Magoc and S. Salzberg. Bioinformatics 27:21 (2011), 2957-63). Next, processed 25 reads were analyzed in Python using the following steps. First, reads were dereplicated and TSO sequence artifacts at the 5′ end of libraries were removed from the sgRNA part of the sequence. Dereplicated reads were then filtered for reads ≤190 bp. Following filtration, dereplicated reads were subsampled to 20,000 reads and collapsed to track the number sequences per sgRNA species. sgRNA species found to be present at ≥1% of the total NGS reads and which were present in all biological replicates were merged. The mean±SD of the proportion of filtered NGS reads versus the total filtered NGS reads were then calculated per sgRNA species. Mean percentages of the sgRNA species NGS reads were then sorted in descending order of the most abundant to the least abundant species. Next, all merged, filtered, and sorted sgRNA species were compared to the provided reference sequence of the sgRNA.

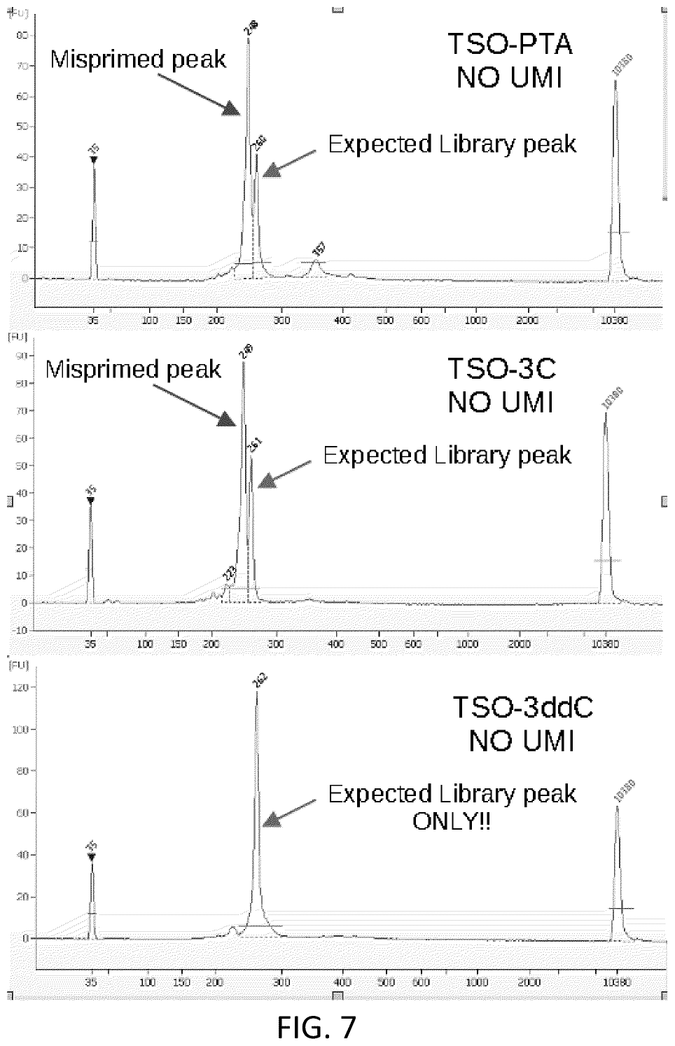

BioAnalyzer electropherograms of the cDNA library with TSO phosphorothioate (TSO-PTA), TSO with three carbon spacers (TSO-3C) and TSO with didieoxyC (TSO-3ddC) were generated. Two major peaks were apparent in the phosphorothioate and three carbon spacer modified TSO oligos with the first peak at approximately 240 nucleotides and the second peak at approximately 260 nucleotides, indicating multiple two different molecules of multiple lengths were present in the mixture (FIG. 7). 260 nucleotides molecules is the expected length for this sgRNA library. The dideoxy cytosine TSO showed only one peak at approximately 260. This mixture showed only the expected library peak with minimal truncated library species.

Neither the phosphorothioate modifications nor the 3C spacer, bypassed the TSO miss-priming, however the dideoxy C TSO had a single and sharp peak with a molecular weight of ˜260 20 bp, which was the expected peak for full-length sgRNA molecules. It showed that the 3ddC modification completely blocks DNA GXL HiFi DNA polymerase.

Blocked TSO Primer Compared with Non-Blocked TSO Primer

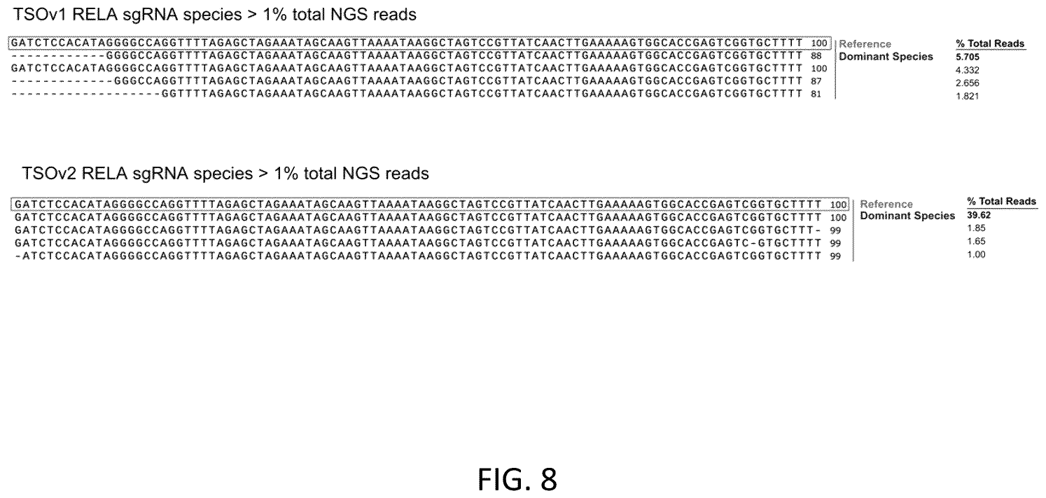

Both TSOv1 (no “blocking” end on the TSO primer) and TSOv2 (with “blocking” end on TSO primer) methods were able to generate data for the RelA guide shown in FIG. 8. In TSOv1, the dominant species sequence did not match the reference RelA sequence. These results were likely library preparation artifacts supported by the Bioanalyzer traces shown in FIG. 7. The TSOv2 method dominant species had a 100% match to the RelA reference sequence. This strongly suggested that the blocked TSO prevents mispriming on internal three G bases.

Blocked TSO Primer Compared with Non-Blocked TSO Primer on Guide RNA without an Internal G-G-G Sequence

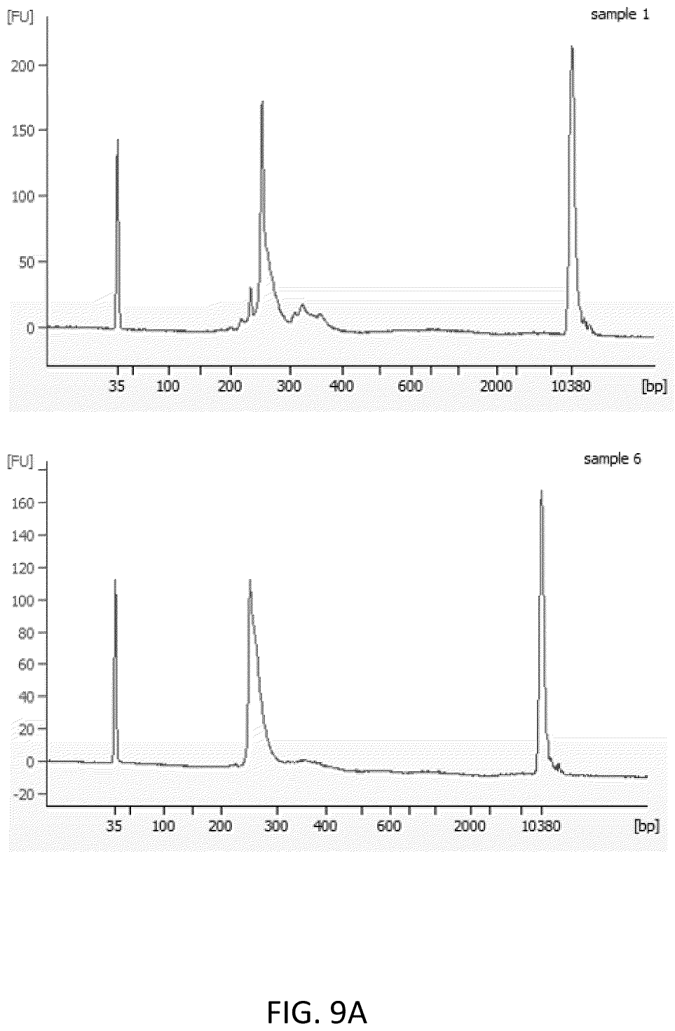

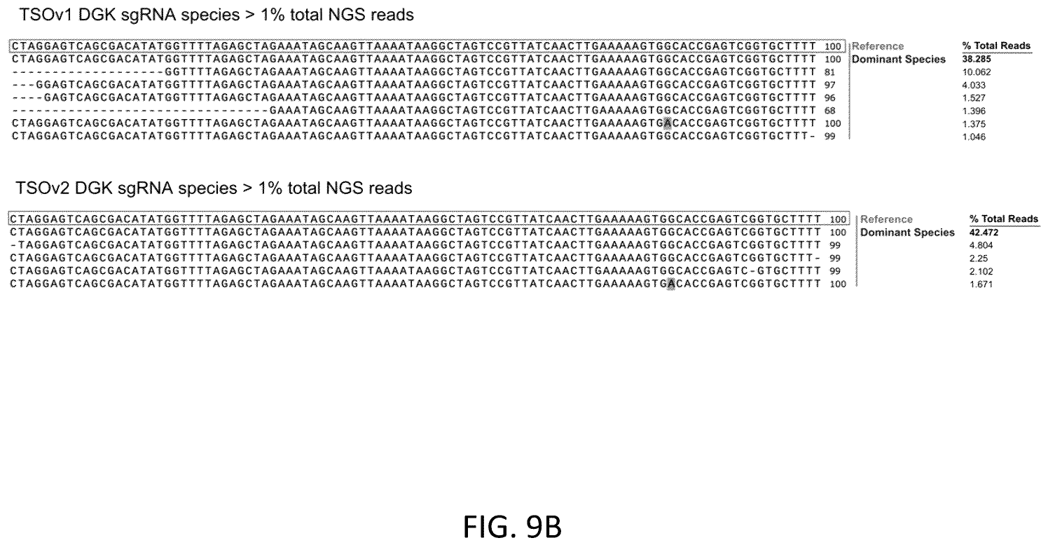

TSOv1 and TSOv2 were also tested on another guide without internal three Gs in the crRNA sequence. In the bioanalyzer traces, there were apparent, small peaks at lower molecular weight for TsOvl (FIG. 9A). These peaks were not present in TSOv2. In TSOv1 sequencing data (FIG. 9B), the dominant species does match the reference species, however, there are multiple truncations in the cRNA sequence involving two or more Gs, suggesting some kind of mis-priming. In TSOv2, the dominant species also matched the reference species. This strongly suggested that the unblocked TSO causes mispriming on internal bases despite the lack the three Gs while the unblocked TSO prevents mispriming on internal bases.

### Example 3

Use of a 3′ Adapter Oligo to Universally Prime Guide RNA Sequences with a Variable 3′ Spacer Region

As guides for Cas12b utilize a guide RNA with a spacer region or variable region at the 3′ end of the guide molecule, cDNA synthesis using a primer specific to the 3′ end of the molecule would be time consuming and expensive as a result of needing to design primer oligos for each 3′ variable spacer region. Additionally, variable GC content causes priming effects. For instance, 20 homo polymers priming annealing temperature among other criteria would introduce bias between molecules and affect the performance of the assay. To bypass these effects, a universal adapter oligonucleotide containing a universal cDNA priming sequence was ligated to the 3′ end of the guide RNA molecule to overcome reverse transcription bias. This method is designated as the “universal method” herein.

HPLC purified RelA guide RNA molecule specific to Cas9 and ACM360 guide RNA molecule specific to Cas12b were used. This RelA molecule has a variable spacer region on the 5′ end of the molecule while this ACM360 molecule has a variable spacer region on the 3′ end of the molecule.

Ligation mix was prepared on ice including 1.8 μl of 100 μM 3′ adapter (5′PNNCTGTCTCTTATACACATCTCCGAGCCCACGAGAC3ddC; SEQ TD NO: 11), 2 μl 10×T4 ligase buffer (New England Biolabs, Boston, MA), 1 μl Recombinant RNase inhibitor (Takara Bio, San Jose, CA), 1 μl T4 ligase 2 (New England Biolabs, Boston, MA), 5 μl 4000 polyethylene glycol (Milipore Sigma, Burlington, MA) and 2.8 μl of molecular grade H2O. Two picomoles of guide RNA were added to the mix in a volume of 6.4 μl. The ligation mixture was incubated at 25° C. for two hours.

First strand cDNA mix was prepared on ice including 0.25 μL of Recombinant RNase Inhibitor (Takara Bio, USA, San Jose, CA) 0.75 μL of 10 mM dNTPs (Takara), 0.1 μL of 10 μM adapter specific oligonucleotide containing an 17 indexing region 10 nucleotides long (Integrated DNA Technologies, Inc., Coralville, Iowa) as indicated in the table below, and 1.875 μL of 40% o PEG (Milipore Sigma). Eight samples total were prepared.

TABLE 5
Example Universal adapter oligo sequences.

| | SEQ ID |
|---|---|
| Universal Adapter Oligo | NO: |
| AATGATACGGCGACCACCGAGATCTACACTCGTGGAGCGTCGTCGGCAGCGTC | 12 |
| AATGATACGGCGACCACCGAGATCTACACCTACAAGATATCGTCGGCAGCGTC | 13 |
| AATGATACGGCGACCACCGAGATCTACACTATAGTAGCTTCGTCGGCAGCGTC | 14 |
| AATGATACGGCGACCACCGAGATCTACACTGCCTGGTGGTCGTCGGCAGCGTC | 15 |
| AATGATACGGCGACCACCGAGATCTACACACATTATCCTTCGTCGGCAGCGTC | 16 |
| AATGATACGGCGACCACCGAGATCTACACGTCCACTTGTTCGTCGGCAGCGTC | 17 |
| AATGATACGGCGACCACCGAGATCTACACTGGAACAGTATCGTCGGCAGCGTC | 18 |
| AATGATACGGCGACCACCGAGATCTACACCCTTGTTAATTCGTCGGCAGCGTC | 19 |

Ligated guide RNA was added in a 9 μl reaction volume to the first strand mix. Each first strand cDNA/TSO mix was incubated for 5 minutes at 70° C. then snap cooled on ice to anneal the primer to the ligated RNA molecule. The second strand mix was prepared with 1.25 μL 100 mM Tris-HCL pH 8.3, 0.1.5 μL 100 mM NaCl, 0.0.5 μL 10 mM GTP, 0.125 100 mM MgCl₂, 0.4 μL 100 mM DTT, 0.2 μL 100 μM template switch oligonucleotide (TSO), and 0.5 Maxima H-minus RT (Thermo Fisher Scientific, Waltham, MA).

Afterward, 5.48 μL of second Strand Mix was added to the snap cooled mixture then incubated using the following protocol to synthesize first and second cDNA strands.

TABLE 6
Example protocol used in Example 3.

| Step # | Temp (° C.) | Time in min. | # Cycles |
|---|---|---|---|
| 1 | 42° C. | 90 min. | 1 |
| 2 | 50° C. | 2 min. | 10 |
| | 42° C. | 2 min. | |
| 3 | 85° C. | 5 min. | 1 |
| 4 | 12° C. | on hold | 1 |

cDNA product was purified with SPRI (MagBio Genomics, Gaithersburg, MD) beads at a 2.5× ratio eluted in 20 μL 1×TE buffer, pH 8.0.

A PCR master mix was prepared using 5 μL 5×GXL Buffer (Takara), 2.5 μL dNTPs (Takara), 0.5 μL GXL Polymerase (Takara) and 13.5 μL molecular biology grade H₂O per reaction. 1 μL 2.5 μM Primer 1, composed of P5 adapter sequence (required to anneal to its complementary oligonucleotide on the Illumina flow cell for sequencing, followed by i5 index sequence (sequence identifier) and 5′ universal linker (Illumina sequencing primer binding site), 1 μL 2.5 μM Primer 2, composed of P7 adapter sequence (required to anneal to its complementary oligo on the Illumina flow cell for sequencing), followed by i7 index sequence (sequence identifier) and 3′ universal linker (Illumina sequencing primer binding site) and 2 μL purified cDNA were added to the reaction. The PCR was cycled using the following program.

TABLE 7
Example PCR protocol used in Example 3.

| Step | Temp | Duration | Cycles |
|---|---|---|---|
| 1 | 98° C. | 30 sec | 1X |
| 2 | 98° C. | 10 sec | 20X |
| | 60° C. | 15 sec | |
| | 65° C. | 30 sec | |
| 3 | 65° C. | 2 min | 1X |
| 4 | 4° C. | hold | 1X |

PCR products were purified using a 1.6× ratio of SPRI and eluted in 20 uL. PCR products were quantified by Qubit HS dsDNA Kit (Thermo Fisher) prior to loading it in the HS dsDNA Bioanalyzer chip (Agilent). Samples were sequenced using an iSeq 2×150 kit at 60 pM concentration.

cDNA product was purified with SPRI (MagBio Genomics, Gaithersburg, MD) beads at a 2.5× ratio eluted in 20 μL 1×TE buffer, pH 8.0. A PCR master mix was prepared using 5 μL 5×GXL Buffer (Takara), 2.5 μL dNTPs (Takara), 0.5 μL GXL Polymerase (Takara) and 10 μL molecular biology grade H₂O per reaction. 1 μL 2.5 μM Primer 1, composed of P5 adapter sequence (required to anneal to its complementary oligo on the Illumina flow cell for sequencing, followed by i5 index sequence (sequence identifier) and 5′ Nextera read 1 site (Illumina sequencing primer binding site), 1 μL 2.5 μM Primer 2, composed of P7 adapter sequence and 5 μL purified cDNA were added to the reaction. The PCR was cycled using the following program.

Raw fastq files were processed using the following steps. Overall high throughput Illumina NGS sequencing quality was assessed using FastQC (Andrews, S. (2010). FastQC: A Quality Control Tool for High Throughput Sequence Data [Online]. Available online at: bioinformatics.babraham.ac.uk/projects/fastqc/per fastq file. Trimmomatic (Bolger, A. M., Lohse, M., & Usadel, B. (2014). Trimmomatic: A flexible trimmer for Illumina Sequence Data.) 20 was used to trim the Illumina sequencing adapters from 5′ and 3′ ends of the reads and to filter for quality reads with a Phred Quality score >Q30. Quality filter and adapter trimmed paired reads were then merged using flash (FLASH: Fast length adjustment of short reads to improve genome assemblies. T. Magoc and S. Salzberg. Bioinformatics 27:21 (2011), 2957-63). Next, processed reads were analyzed in Python using the following steps. First, reads were dereplicated and TSO sequence artifacts at the 5′ end of libraries and the two degenerate 3′ adapter bases at the 3′ end of the libraries were removed from the sgRNA part of the sequence. Dereplicated reads were then filtered for reads ≤190 bp. Following filtration, dereplicated reads were subsampled to 20,000 reads and collapsed to track the number sequences per sgRNA species. sgRNA species found to be present at ≥1% of the total NGS reads and which were present in all biological replicates were merged. The mean±SD of the proportion of filtered NGS reads versus the total filtered NGS reads were then calculated per sgRNA species. Mean percentages of the sgRNA species NGS reads were then sorted in descending order of the most abundant to the least abundant species. Next, all merged, filtered, and sorted sgRNA species were compared to the provided reference sequence of the sgRNA.

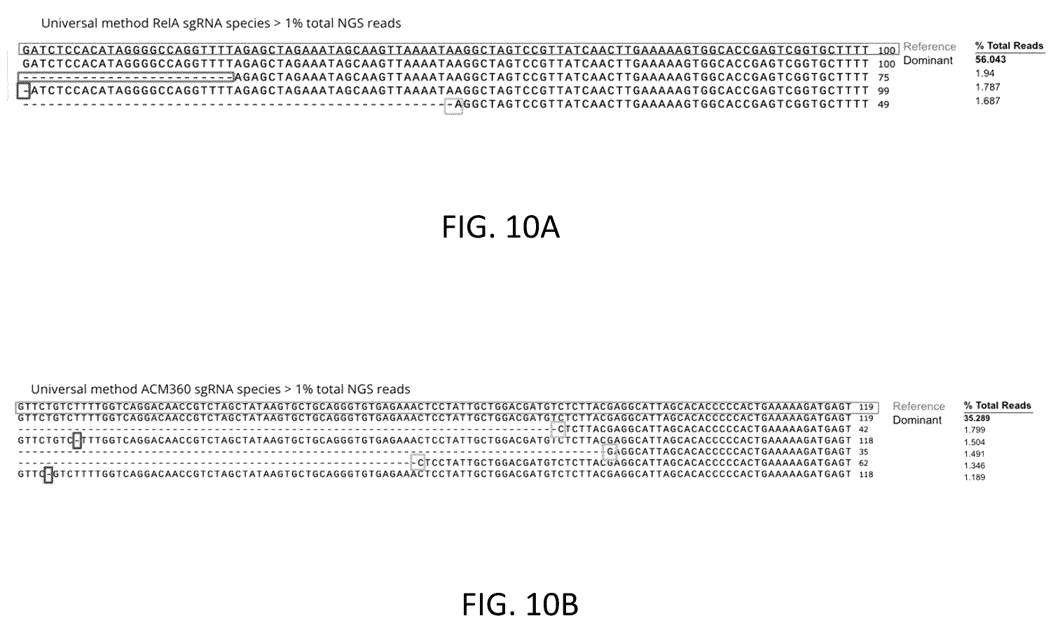

Sequencing reads were aligned to the RelA reference sequence and sorted in order of abundance (FIG. 10A). The most common species found in the sequencing reads was a 100% match to the reference RelA sequence. This strongly suggests that the universal sequencing platform can verify the sequence of a guide with a 5′ variable spacer region.

Sequencing reads were aligned to the ACM360 reference sequence and sorted in order of abundance (FIG. 10B). The most common species found in the sequencing reads was a 100% match to the ACM360 reference sequence. This strongly suggests that the universal sequencing platform can verify the sequence of a guide with a 3′ variable spacer region.

### Example 4

### Introduction of UMI During the Ligation to the 3′ End of RNA Sample for UMI

RelA guide RNA molecule specific to Cas9 and its single-nucleotide variants were used. These RNA molecules were chemically synthesized and purified by reverse-phase HPLC (RP-HPLC).

Ligation mix was prepared on ice including 0.45 μL of 100 μM 3′ adapter with UMI (5′rAppNNATCNNNNNNNNNNCTGTCTCTTATACACATCTCCGAGCCCACGAGAC3ddC SEQ ID NO: 20), 2 μL lOX T4 ligase buffer (New England Biolabs, Boston, MA), 1 μL Recombinant RNase inhibitor (Takara Bio, San Jose, CA), 1 μL T4 ligase 2 (New England Biolabs, Boston, MA), 5 μL 40% polyethylene glycol (Milipore Sigma, Burlington, MA), and 5.55 μL of molecular grade H₂O. Three picomoles of RNA mixture samples were added to the mix in a volume of 5 μL. The ligation mixture was incubated at 20° C. for four hours.

The rest of the experimental procedures were the same as those in Example 3, discussed earlier.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

What is claimed is:

1. A method for determining a sequence of a guide RNA, comprising:
a. providing a guide RNA from a sample;
b. preparing a first and second strand mix comprising:
i. the guide RNA;
ii. DNA primer that hybridizes to the 3′ end of the guide RNA;
iii. dNTPs;
iv. a template switching oligonucleotide (TSO) comprising a modification at the 3′ terminal end; and
c. incubating the first strand mix and the second strand mix under conditions to allow synthesis of cDNA;
d. purifying the cDNA;
e. amplifying the cDNA;
f. purifying the amplified cDNA; and
g. sequencing the amplified cDNA.

2. The method of claim 1, wherein the modification is a 3-dideoxycytosine (3ddC).

3. The method of any one of the preceding claims, further comprising the step of ligating a universal adapter oligonucleotide to the guide RNA between steps a) and b).

4. The method of any one of the preceding claims, further comprising comparing the sequences with a reference guide RNA sequence.

5. The method of any one of the preceding claims, wherein the cDNA is amplified by PCR.

6. The method of any one of the preceding claims, wherein the sequencing comprises NGS.

7. The method of any one of the preceding claims, wherein the amplified cDNA is cloned into expression vectors for Sanger sequencing.

8. The method of any one of the preceding claims, wherein the guide RNA is about 40 to about 120 nucleotides in length.

9. The method of any one of the preceding claims, wherein a sequence is determined for the entire guide RNA.

10. The method of any one of the preceding claims, wherein the 3′ end of the TSO comprises the sequence -GGG-3ddC or -rGrGrG-3ddC.

11. The method of any one of the preceding claims, wherein the 3′ end of the TSO comprises the sequence -GGGG-3ddC or -GrGrGrG-3ddC.

12. A method for preparing a cDNA copy of a guide RNA, comprising:
a. providing a guide RNA from a sample;
b. preparing a first and second strand mix comprising:
i. the guide RNA;
ii. DNA primer that hybridizes to the 3′ end of the guide RNA;
iii. dNTPs;
iv. a template switching oligonucleotide (TSO) comprising a modification at the 3′ end;
v. a reverse transcriptase; and
c. incubating the first strand mix and the second strand mix under conditions to allow synthesis of cDNA.

13. The method of claim 12, wherein the guide RNA is about 40 to about 120 nucleotides in length.

14. The method of claims 11-13, wherein a sequence is determined for the entire guide RNA.

15. The method of claims 11-14, wherein the 3′ end modification is 3-dideoxycytosine (3ddC).

16. The method of claims 11-15, wherein the 3′ end of the TSO comprises the sequence -GGG-3ddC or -rGrGrG-3ddC.

17. The method of claims 11-16, wherein the 3′ end of the TSO comprises the sequence -GGGG-3ddC or -GrGrGrG-3ddC.

18. A template switching oligonucleotide (TSO) comprising a modification at the 3′ end that inhibits DNA polymerase activity.

19. The TSO of claim 18, wherein the modification is a 3-dideoxycytosine (3ddC).

20. The TSO of claim 19, wherein the 3′ end of the TSO comprises the sequence -GGG-3ddC or -rGrGrG-3ddC.

21. The TSO of claim 19, wherein the 3′ end of the TSO comprises the sequence -GGGG-3ddC or -GrGrGrG-3ddC.

22. A cDNA preparation mixture, comprising:
a. a guide RNA;
b. a DNA primer that hybridizes to the 3′ end of the guide RNA;
c. a template switching oligonucleotide (TSO) comprising a 3-Dideoxy Cytosine (3ddC) modification at the 3′ end; and
d. optionally dNTPs and/or a reverse transcriptase.

23. A kit for generating cDNA copies of a guide RNA, comprising a template switching oligonucleotide (TSO) that has a 3-dideoxycytosine (3ddC) modification at the 3′ end.

24. The kit of claim 23, further comprising a DNA primer that hybridizes to the 3′ end of a guide RNA or universal adapter oligonucleotide.

25. The kit of claim 23, further comprising dNTPs.

26. The kit of claim 23, further comprising a reverse transcriptase.

27. A method for assessing the quality of a guide RNA in a sample, comprising:
a. providing a guide RNA from the sample;
b. preparing a first and second strand mix comprising:
i. the guide RNA;
ii. DNA primer that hybridizes to the 3′ end of the guide RNA;
iii. dNTPs; and
iv. a template switching oligonucleotide (TSO) comprising a modification at the 3′ end;
c. incubating the first strand mix and the second strand mix under conditions to allow synthesis of cDNA;
d. sequencing the amplified cDNA; and
e. comparing the sequence of the amplified cDNA to a reference guide RNA sequence.

28. The method of claim 27, wherein the modification is a 3-dideoxycytosine (3ddC).

29. The method of any one of claims 27-28, further comprising the step of ligating a universal adapter oligonucleotide to the guide RNA between steps a) and b).

30. The method of any one of claims 27-29, further comprising comparing the sequences with a reference guide RNA sequence.

31. The method of any one of claims 27-30, wherein the cDNA is amplified by PCR.

32. The method of any one of claims 27-31, wherein the sequencing comprises NGS.

33. The method of any one of claims 27-32, wherein the amplified cDNA is cloned into expression vectors for Sanger sequencing.

34. The method of any one of claims 27-33, wherein the guide RNA is about 40 to about 120 nucleotides in length.

35. The method of any one of claims 27-34, wherein a sequence is determined for the entire guide RNA.

36. The method of any one of claims 27-35, wherein the 3′ end of the TSO comprises the sequence -GGG-3ddC or -rGrGrG-3ddC.

37. The method of any one of claims 27-36, wherein the 3′ end of the TSO comprises the sequence -GGGG-3ddC or -GrGrGrG-3ddC.

38. A method for assessing the quality of RNA in a sample, comprising:
a. providing an RNA molecule from the sample;
b. preparing a first and second strand mix comprising:
i. the RNA;
ii. DNA primer that hybridizes to the 3′ end of the RNA;
iii. dNTPs; and
iv. a template switching oligonucleotide (TSO) comprising a modification at the 3′ end;
c. incubating the first strand mix and the second strand mix under conditions to allow synthesis of cDNA;
d. sequencing the amplified cDNA; and
e. comparing the sequence of the amplified cDNA to a reference RNA sequence.

39. The method of claim 38, wherein the modification is a 3-dideoxycytosine (3ddC).

40. The method of any one of claims 38-39, further comprising the step of ligating a universal adapter oligonucleotide to the RNA between steps a) and b).

41. The method of claim 40, wherein a plurality of unique molecular identifier (UMI) sequences are added to the 3′-end of the RNA during ligation.

42. The method of any one of claims 38-41, wherein the first and second strand mix further comprises a reverse transcriptase.

43. The method of claim 42, wherein a plurality of unique molecular identifier (UMI) sequences are added to the cDNA via reverse transcription during cDNA synthesis.

44. The method of any one of claims 41-43, wherein each UMI sequence in the plurality of UMI sequences is at least 21 nucleotides in length.

45. The method of any one of claims 41-44, wherein each UMI sequence in the plurality of UMI sequences is at least 10 nucleotides in length.

46. The method of any one of claims 41-45, wherein the length of length of each UMI sequence in the plurality of UMI sequences is determined by a concentration of the RNA in the sample.

47. The method of any one of claims 41-46, wherein the method further comprises determining the percentage of recovered RNA species based on the plurality of UMI sequences.

48. The method of any one of claims 41-47, wherein the method further comprises determining the percentage of each UMI sequence in the plurality of UMI sequences with unique RNA sequence reads.

49. The method of any one of claims 41-47, wherein the number of UMI sequences in the plurality of UMI sequences is about the number of RNA molecules in the sample.

50. The method of any one of claims 38-47, further comprising comparing the sequences with a reference guide RNA sequence.

51. The method of any one of claims 38-50, wherein the cDNA is amplified by PCR.

52. The method of any one of claims 38-51, wherein the sequencing comprises NGS.

53. The method of any one of claims 38-52, wherein the amplified cDNA is cloned into expression vectors for Sanger sequencing.

54. The method of any one of claims 38-53, wherein the guide RNA is about 40 to about 120 nucleotides in length.

55. The method of any one of claims 38-54, wherein a sequence is determined for the entire guide RNA.

56. The method of any one of claims 38-55, wherein the 3′ end of the TSO comprises the sequence -GGG-3ddC or -rGrGrG-3ddC.

57. The method of any one of claims 38-56, wherein the 3′ end of the TSO comprises the sequence -GGGG-3ddC or -GrGrGrG-3ddC.

58. A method for determining a sequence of an RNA molecule, comprising:
a. providing the RNA from a sample;
b. preparing a first and second strand mix comprising:
i. the RNA molecule;
ii. DNA primer that hybridizes to the 3′ end of the RNA molecule;
iii. dNTPs;
iv. a template switching oligonucleotide (TSO) comprising a modification at the 3′ terminal end; and
c. incubating the first strand mix and the second strand mix under conditions to allow synthesis of cDNA;
d. sequencing the cDNA.

59. A method for determining a sequence of an RNA molecule, comprising:
a. providing the RNA from a sample;
b. preparing a ligation mix comprising the RNA molecule, an adapter oligonucleotide containing adapter sequence that is covalently ligated to the 3′ end of the RNA molecule; RNA ligase; and a template switching oligonucleotide (TSO) comprising a modification at the 3′ terminal end;
c. incubating the ligation mix under conditions to allow ligation of the DNA oligonucleotide to the 3′ end of the RNA molecule;
d. purifying the ligated RNA molecule;
e. preparing a first and second strand mix comprising: the ligated RNA molecule, DNA primer that hybridizes to the adapter sequence in the ligated DNA oligonucleotide, dNTPs, and a template switching oligonucleotide (TSO) comprising a modification at the 3′ terminal end; and
f. incubating the first strand mix and the second strand mix under conditions to allow synthesis of cDNA; and
g. sequencing the cDNA.

60. The method of claim 58 or 59, wherein the modification is a 3-dideoxycytosine (3ddC).

61. The method of claim 58 or 59, wherein the RNA molecule is a guide RNA.


SEQUENCE LISTING (44 sequences)

SEQ ID NO: 1 - DNA, 36 bp, synthetic construct
  source 1..36: mol_type = other DNA
nnctgtctcttatacacatctccgagcccacgagac

SEQ ID NO: 2 - DNA, 37 bp, synthetic construct
  source 1..37: mol_type = other DNA
  modified_base 37: mod_base = OTHER; note = 3-dideoxycytosine
nnctgtctcttatacacatctccgagcccacgagacc

SEQ ID NO: 3 - DNA, 34 bp, synthetic construct
  source 1..34: mol_type = other DNA
ctgtctcttatacacatctccgagcccacgagac

SEQ ID NO: 4 - DNA, 35 bp, synthetic construct
  source 1..35: mol_type = other DNA
  modified_base 35: mod_base = OTHER; note = 3-dideoxycytosine
ctgtctcttatacacatctccgagcccacgagacc

SEQ ID NO: 5 - DNA, 66 bp, synthetic construct
  source 1..66: mol_type = other DNA
nnatcacatgtcagtcatctacgatcgatcatctgtctcttatacacatctccgagcccacgagac

SEQ ID NO: 6 - DNA, 36 bp, synthetic construct
  source 1..36: mol_type = other DNA
  modified_base 34..36: mod_base = OTHER; note = guanosine
tcgtcggcagcgtcagatgtgtataagagacagggg

SEQ ID NO: 7 - DNA, 50 bp, synthetic construct
  source 1..50: mol_type = other DNA
tctcgtgggctcggagatgtgtataagagacagaaaagcaccgactcggt

SEQ ID NO: 8 - DNA, 36 bp, synthetic construct; identical to SEQ ID NO: 6
  source 1..36: mol_type = other DNA
  modified_base 34..36: mod_base = OTHER; note = guanosine
  modified_base 36: mod_base = OTHER; note = phosphorothiate

SEQ ID NO: 9 - DNA, 36 bp, synthetic construct; identical to SEQ ID NO: 6
  source 1..36: mol_type = other DNA
  misc_feature 36: note = 3 carbon spacer
  modified_base 34..36: mod_base = OTHER; note = guanosine

SEQ ID NO: 10 - DNA, 37 bp, synthetic construct
  source 1..37: mol_type = other DNA
  modified_base 37: mod_base = OTHER; note = 3-dideoxycytosine
  modified_base 34..36: mod_base = OTHER; note = guanosine
tcgtcggcagcgtcagatgtgtataagagacaggggc

SEQ ID NO: 11 - DNA, 37 bp, synthetic construct; identical to SEQ ID NO: 2
  source 1..37: mol_type = other DNA
  modified_base 37: mod_base = OTHER; note = 3-dideoxycytosine
  misc_feature 1: note = 5'P

SEQ ID NO: 12 - DNA, 53 bp, synthetic construct
  source 1..53: mol_type = other DNA
aatgatacggcgaccaccgagatctacactcgtggagcgtcgtcggcagcgtc

SEQ ID NO: 13 - DNA, 53 bp, synthetic construct
  source 1..53: mol_type = other DNA
aatgatacggcgaccaccgagatctacacctacaagatatcgtcggcagcgtc

SEQ ID NO: 14 - DNA, 53 bp, synthetic construct; identical to SEQ ID NO: 13
  source 1..53: mol_type = other DNA

SEQ ID NO: 15 - DNA, 53 bp, synthetic construct
  source 1..53: mol_type = other DNA
aatgatacggcgaccaccgagatctacactgcctggtggtcgtcggcagcgtc

SEQ ID NO: 16 - DNA, 53 bp, synthetic construct
  source 1..53: mol_type = other DNA
aatgatacggcgaccaccgagatctacacacattatccttcgtcggcagcgtc

SEQ ID NO: 17 - DNA, 53 bp, synthetic construct
  source 1..53: mol_type = other DNA
aatgatacggcgaccaccgagatctacacgtccacttgttcgtcggcagcgtc

SEQ ID NO: 18 - DNA, 53 bp, synthetic construct
  source 1..53: mol_type = other DNA
aatgatacggcgaccaccgagatctacactggaacagtatcgtcggcagcgtc

SEQ ID NO: 19 - DNA, 53 bp, synthetic construct
  source 1..53: mol_type = other DNA
aatgatacggcgaccaccgagatctacacccttgttaattcgtcggcagcgtc

SEQ ID NO: 20 - DNA, 51 bp, synthetic construct
  source 1..51: mol_type = other DNA
  misc_feature 1: note = 5'rApp
  modified_base 51: mod_base = OTHER; note = 3-dideoxycytosine
annatcnnnnnnnnnnctgtctcttatacacatctccgagcccacgagacc

SEQ ID NO: 21 - DNA, 100 bp, synthetic construct
  source 1..100: mol_type = other DNA
gatctccacataggggccaggttttagagctagaaatagcaagttaaaataaggctagtccgttatcaacttgaaaaagtggcaccgagtcggtgctttt

SEQ ID NO: 22 - DNA, 88 bp, synthetic construct
  source 1..88: mol_type = other DNA
ggggccaggttttagagctagaaatagcaagttaaaataaggctagtccgttatcaacttgaaaaagtggcaccgagtcggtgctttt

SEQ ID NO: 23 - DNA, 87 bp, synthetic construct
  source 1..87: mol_type = other DNA
gggccaggttttagagctagaaatagcaagttaaaataaggctagtccgttatcaacttgaaaaagtggcaccgagtcggtgctttt

SEQ ID NO: 24 - DNA, 81 bp, synthetic construct
  source 1..81: mol_type = other DNA
ggttttagagctagaaatagcaagttaaaataaggctagtccgttatcaacttgaaaaagtggcaccgagtcggtgctttt

SEQ ID NO: 25 - DNA, 99 bp, synthetic construct
  source 1..99: mol_type = other DNA
gatctccacataggggccaggttttagagctagaaatagcaagttaaaataaggctagtccgttatcaacttgaaaaagtggcaccgagtcggtgcttt

SEQ ID NO: 26 - DNA, 99 bp, synthetic construct
  source 1..99: mol_type = other DNA
gatctccacataggggccaggttttagagctagaaatagcaagttaaaataaggctagtccgttatcaacttgaaaaagtggcaccgagtcgtgctttt

SEQ ID NO: 27 - DNA, 99 bp, synthetic construct
  source 1..99: mol_type = other DNA
atctccacataggggccaggttttagagctagaaatagcaagttaaaataaggctagtccgttatcaacttgaaaaagtggcaccgagtcggtgctttt

SEQ ID NO: 28 - DNA, 100 bp, synthetic construct
  source 1..100: mol_type = other DNA
ctaggagtcagcgacatatggttttagagctagaaatagcaagttaaaataaggctagtccgttatcaacttgaaaaagtggcaccgagtcggtgctttt

SEQ ID NO: 29 - DNA, 81 bp, synthetic construct; identical to SEQ ID NO: 24
  source 1..81: mol_type = other DNA

SEQ ID NO: 30 - DNA, 97 bp, synthetic construct
  source 1..97: mol_type = other DNA
ggagtcagcgacatatggttttagagctagaaatagcaagttaaaataaggctagtccgttatcaacttgaaaaagtggcaccgagtcggtgctttt

SEQ ID NO: 31 - DNA, 96 bp, synthetic construct
  source 1..96: mol_type = other DNA
gagtcagcgacatatggttttagagctagaaatagcaagttaaaataaggctagtccgttatcaacttgaaaaagtggcaccgagtcggtgctttt

SEQ ID NO: 32 - DNA, 68 bp, synthetic construct
  source 1..68: mol_type = other DNA
gaaatagcaagttaaaataaggctagtccgttatcaacttgaaaaagtggcaccgagtcggtgctttt

SEQ ID NO: 33 - DNA, 100 bp, synthetic construct
  source 1..100: mol_type = other DNA
ctaggagtcagcgacatatggttttagagctagaaatagcaagttaaaataaggctagtccgttatcaacttgaaaaagtgacaccgagtcggtgctttt

SEQ ID NO: 34 - DNA, 99 bp, synthetic construct
  source 1..99: mol_type = other DNA
ctaggagtcagcgacatatggttttagagctagaaatagcaagttaaaataaggctagtccgttatcaacttgaaaaagtggcaccgagtcggtgcttt

SEQ ID NO: 35 - DNA, 99 bp, synthetic construct
  source 1..99: mol_type = other DNA
taggagtcagcgacatatggttttagagctagaaatagcaagttaaaataaggctagtccgttatcaacttgaaaaagtggcaccgagtcggtgctttt

SEQ ID NO: 36 - DNA, 99 bp, synthetic construct
  source 1..99: mol_type = other DNA
ctaggagtcagcgacatatggttttagagctagaaatagcaagttaaaataaggctagtccgttatcaacttgaaaaagtggcaccgagtcgtgctttt

SEQ ID NO: 37 - DNA, 75 bp, synthetic construct
  source 1..75: mol_type = other DNA
agagctagaaatagcaagttaaaataaggctagtccgttatcaacttgaaaaagtggcaccgagtcggtgctttt

SEQ ID NO: 38 - DNA, 49 bp, synthetic construct
  source 1..49: mol_type = other DNA
aggctagtccgttatcaacttgaaaaagtggcaccgagtcggtgctttt

SEQ ID NO: 39 - DNA, 119 bp, synthetic construct
  source 1..119: mol_type = other DNA
gttctgtcttttggtcaggacaaccgtctagctataagtgctgcagggtgtgagaaactcctattgctggacgatgtctcttacgaggcattagcacacccccactgaaaaagatgagt

SEQ ID NO: 40 - DNA, 42 bp, synthetic construct
  source 1..42: mol_type = other DNA
ctcttacgaggcattagcacacccccactgaaaaagatgagt

SEQ ID NO: 41 - DNA, 118 bp, synthetic construct
  source 1..118: mol_type = other DNA
gttctgtctttggtcaggacaaccgtctagctataagtgctgcagggtgtgagaaactcctattgctggacgatgtctcttacgaggcattagcacacccccactgaaaaagatgagt

SEQ ID NO: 42 - DNA, 35 bp, synthetic construct
  source 1..35: mol_type = other DNA
gaggcattagcacacccccactgaaaaagatgagt

SEQ ID NO: 43 - DNA, 62 bp, synthetic construct
  source 1..62: mol_type = other DNA
ctcctattgctggacgatgtctcttacgaggcattagcacacccccactgaaaaagatgagt

SEQ ID NO: 44 - DNA, 118 bp, synthetic construct
  source 1..118: mol_type = other DNA
gttcgtcttttggtcaggacaaccgtctagctataagtgctgcagggtgtgagaaactcctattgctggacgatgtctcttacgaggcattagcacacccccactgaaaaagatgagt